(19) United States
(12) Patent Application Publication
(10) Pub. No.: US 2007/0167456 A1
(43) Pub. Date: Jul. 19, 2007

(54) Mercaptoimidazoles as ccr2 receptor antagonists

(76) Inventors: Guy Rosalia Eugeen Lommen, Berlaar (BE); Gustaaf Maria Boeckx, Oud-Turnhout (BE)

Correspondence Address: Janssen Pharmaceutica N V, Turnhoutseweg 30, Beerse, B-2340, BE

(21) Appl. No.: 11/572,770

(22) PCT Filed: Aug. 10, 2005
(86) PCT No.: PCT/EP05/53937

§ 371 Date: Jan. 26, 2007

(30) Foreign Application Priority Data
Aug. 11, 2004 (EP) 04103875.3

(51) Int. Cl. A61K 31/506; A61K 31/498; A61K 31/4439; A61K 31/427; A61K 31/433; A61K 31/4245; A61K 31/422; A61K 31/4196; A61K 31/4178; C07D 417/02; C07D 413/02; C07D 403/02

(52) U.S. Cl. 514/249; 514/256; 514/341; 514/362; 514/364; 514/365; 514/374; 514/378; 514/388; 514/381; 514/383; 544/353; 544/333; 546/272.1; 548/253; 548/134; 548/136; 548/143; 548/202; 548/235; 548/247; 548/311.1

(57) ABSTRACT

The present invention relates to a compound of formula (I) 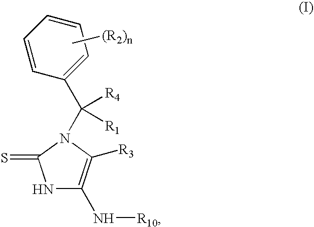
a N-oxide, a pharmaceutically acceptable addition salt, a quaternary amine and a stereochemically isomeric form thereof, wherein R₁ represents hydrogen, C₁₋₆alkyl, C₃₋₇cycloalkyl, C₁₋₆alkyloxyC₁₋₆alkyl, di(C₁₋₆alkyl)aminoC₁₋₆alkyl, aryl or heteroaryl; each R₂ independently represents halo, C₁₋₆alkyl, C₁₋₆alkyloxy, C₁₋₆alkylthio, polyhaloC₁₋₆alkyl, polyhaloC₁₋₆alkyloxy, cyano, aminocarbonyl, amino, mono- or di(C₁₋₄alkyl)amino, nitro, aryl or aryloxy; R₃ represents cyano, C(═O)—O—R₅, C(═O)—NR₆ₐR_{6b} or C(═O)—R₇; or a cyclic ring system; R₄ represents hydrogen or C₁₋₆alkyl; n is 1, 2, 3, 4 or 5; R₁₀ represents hydrogen, C₁₋₆alkyl, C₁₋₆alkylcarbonyl, C₁₋₆alkyloxycarbonyl, arylcarbonyl, heteroarylcarbonyl, —C(═O)—NH—R₅, —C(═S)—NH—R₅ or —S(═O)₂—R₅. The invention also relates to processes for preparing the compounds of formula (I), their use as CCR2 antagonists and pharmaceutical compositions comprising them.

The present invention concerns mercaptoimidazole derivatives having CCR2 receptor antagonistic properties. The invention further relates to methods for their preparation and pharmaceutical compositions comprising them. The invention also relates to the use of said compounds for the manufacture of a medicament for the prevention or the treatment of diseases mediated through activation of the CCR2 receptor, in particular the CCR2B receptor.

WO 02/066458 describes 2-thio-substituted imidazole derivatives having immunomodulating and/or inhibiting activity on the release of cytokines, especially TNF-α and IL-β.

FR 1,487,326 relates to thio-imidazole derivatives useful as analgetic and for its vasodilatation activity.

FR 6,751 M describes thio-imidazole derivatives as sedatives and analgesics.

U.S. Pat. No. 3,850,944 describes 2-mercapto-5-(3-pyridyl)-imidazole derivatives having antiinflammatory activity.

EP 0,277,384 describes 1H-imidazole-5-carboxylic acid derivatives for controlling weeds.

The compounds of the invention differ from the prior art compounds in structure, in their pharmacological activity and/or pharmacological potency.

One aspect of the present invention relates to a compound of formula 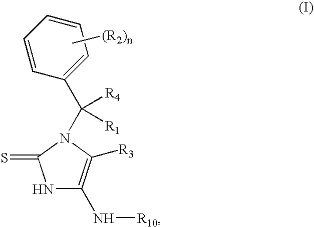
a N-oxide, a pharmaceutically acceptable addition salt, a quaternary amine or a stereochemically isomeric form thereof, wherein
R₁ represents hydrogen, C₁₋₆alkyl, C₃₋₇cycloalkyl, C₁₋₆alkyloxyC₁₋₆alkyl, di(C₁₋₆alkyl)aminoC₁₋₆alkyl, aryl or heteroaryl;
each R₂ independently represents halo, C₁₋₆alkyl, C₁₋₆alkyloxy, C₁₋₆alkylthio, polyhaloC₁₋₆alkyl, polyhaloC₁₋₆alkyloxy, cyano, aminocarbonyl, amino, mono- or di(C₁₋₄alkyl)amino, nitro, aryl or aryloxy;
R₃ represents cyano, C(═O)—O—R₅, C(═O)—NR₆ₐR_{6b} or C(═O)—R₇; or a cyclic ring system selected from 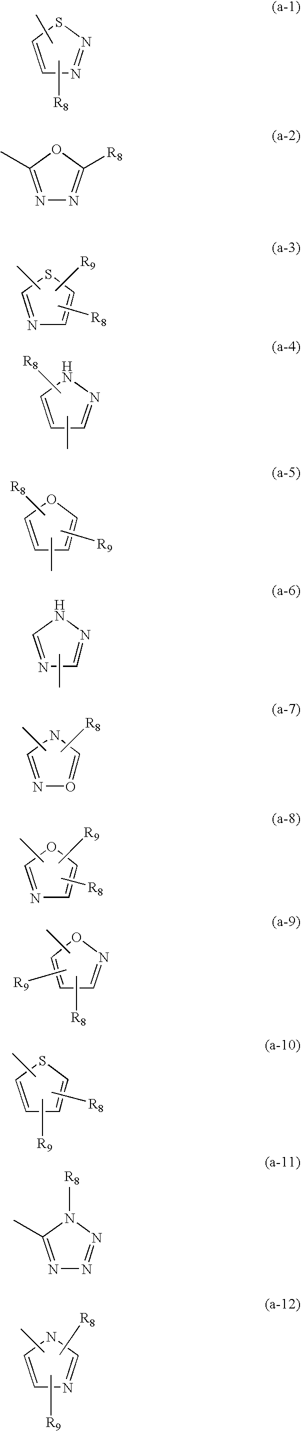 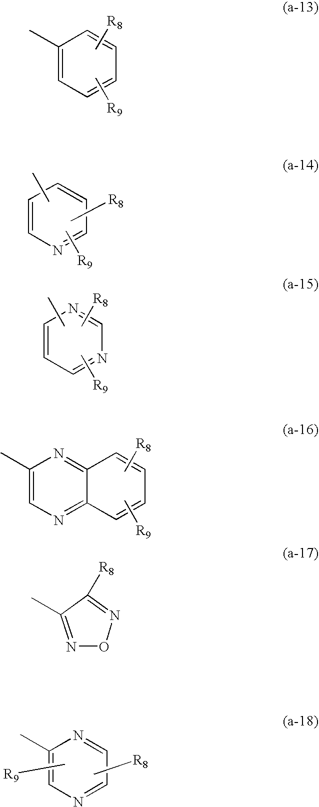
R₄ represents hydrogen or C₁₋₆alkyl;
R₅ represents hydrogen, C₁₋₆alkyl, hydroxyC₁₋₆alkyl, C₂₋₆alkenyl, C₂₋₆alkynyl, polyhaloC₁₋₆alkyl, C₁₋₆alkyloxyC₁₋₆alkyl optionally substituted with C₁₋₆alkyloxy, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl, aryl or arylC₁₋₆alkyl;
R₆ₐ and R_{6b} each independently represent hydrogen, C₁₋₆alkyl, amino, mono- or di(C₁₋₄alkyl)amino, arylNH—, aminoC₁₋₆alkyl, mono- or di(C₁₋₄alkyl)amino-C₁₋₆alkyl, C₁₋₆alkylcarbonylamino, aminocarbonylamino, C₁₋₆alkyloxy, carbonylamino or hydroxyC₁₋₆alkyl; or
R₆ₐ and R_{6b} taken together with the nitrogen to which they are attached form pyrrolidinyl, imidazolidinyl, pyrazolidinyl, piperidinyl, piperazinyl, morpholinyl, thiomorpholinyl or piperazinyl substituted with C₁₋₆alkyl;
R₇ represents hydrogen, C₁₋₆alkyl, hydroxyC₁₋₆alkyl, C₂₋₆alkenyl, C₂₋₆alkynyl, polyhaloC₁₋₆alkyl, C₁₋₆alkyloxyC₁₋₆alkyl, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl, aryl or heteroaryl;
each R₈ independently represents hydrogen, halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, hydroxyC₁₋₆alkylamino, aryl, aryloxy, piperidinyl, piperidinylamino, morpholinyl, piperazinyl or nitro;
each R₉ independently represents hydrogen, halo or C₁₋₆alkyl;
R₁₀ represents hydrogen, C₁₋₆alkyl, C₁₋₆alkylcarbonyl, C₁₋₆alkyloxycarbonyl, arylcarbonyl, heteroarylcarbonyl, —C(═O)—NH—R₅, —C(═S)—NH—R₅ or —S(═O)₂—R₅;
n is 1, 2, 3, 4 or 5;
aryl represents phenyl or phenyl substituted with one, two, three, four or five substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, phenyloxy or nitro;
heteroaryl represents pyrrolidinyl, tetrahydrofuranyl, imidazolidinyl, pyrazolidinyl, pyrrolinyl, imidazolinyl, pyrazolinyl, furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, piperidinyl, piperazinyl, morpholinyl, thiomorpholinyl, each of said heterocycles optionally being substituted with one or two substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, nitro or arylC₁₋₆alkyl.

The present invention also relates to the use of a compound of formula (I) for the manufacture of a medicament for preventing or treating a disease, in particular for treating a disease, mediated through activation of the CCR2 receptor, in particular for preventing or treating an inflammatory disease.

As used hereinbefore or hereinafter C₁₋₄alkyl as a group or part of a group defines straight or branched chain saturated hydrocarbon radicals having from 1 to 4 carbon atoms such as methyl, ethyl, propyl, 1-methylethyl, butyl; C₁₋₆alkyl as a group or part of a group defines straight or branched chain saturated hydrocarbon radicals having from 1 to 6 carbon atoms such as the group defined for C₁₋₄alkyl and pentyl, hexyl, 2-methylbutyl and the like; C₃₋₇cycloalkyl is generic to cyclopropyl, cyclobutyl, cyclopentyl, cyclohexyl and cycloheptyl; C₂₋₆alkenyl defines straight and branched chain hydrocarbon radicals having from 2 to 6 carbon atoms containing a double bond such as ethenyl, propenyl, butenyl, pentenyl, hexenyl and the like; C₂₋₆alkynyl defines straight and branched chain hydrocarbon radicals having from 2 to 6 carbon atoms containing a triple bond such as ethynyl, propynyl, butynyl, pentynyl, hexynyl and the like.

As used hereinbefore, the term (═O) forms a carbonyl moiety when attached to a carbon atom, a sulfoxide moiety when attached to a sulfur atom and a sulfonyl moiety when two of said terms are attached to a sulfur atom.

The term halo is generic to fluoro, chloro, bromo and iodo. As used in the foregoing or hereinafter, polyhaloC₁₋₆alkyl as a group or part of a group is defined as mono- or polyhalosubstituted C₁₋₆alkyl, for example methyl with one or more fluoro atoms, for example, difluoromethyl or trifluoromethyl, 1,1-difluoro-ethyl and the like. In case more than one halogen atoms are attached to an alkyl group within the definition of polyhaloC₁₋₆alkyl, they may be the same or different.

The term heteroaryl, e.g. in the definition of R₁, R₇ or R₁₀, is meant to include all the possible isomeric forms of the heterocycles, for instance, pyrrolyl comprises 1H-pyrrolyl and 2H-pyrrolyl.

The aryl, heteroaryl or cyclic ring systems listed in the definitions of the substituents of the compounds of formula (I) (see for instance R₁, R₅ and R₃) as mentioned hereinabove or hereinafter may be attached to the remainder of the molecule of formula (I) through any ring carbon or heteroatom as appropriate, if not otherwise specified. Thus, for example, when heteroaryl is imidazolyl, it may be 1-imidazolyl, 2-imidazolyl, 4-imidazolyl and the like.

When any variable (e.g. R₅) occurs more than one time in any constituent, each definition is independent.

Lines drawn from substituents into ring systems indicate that the bond may be attached to any of the suitable ring atoms. When the lines are drawn into bicyclic ring systems, it indicates that the bond may be attached to any of the suitable ring atoms of any one of the two cycles of the bicyclic ring system.

For therapeutic use, salts of the compounds of formula (I) are those wherein the counterion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not are included within the ambit of the present invention.

The pharmaceutically acceptable addition salts as mentioned hereinabove are meant to comprise the therapeutically active non-toxic acid addition salt forms which the compounds of formula (I) are able to form. The latter can conveniently be obtained by treating the base form with such appropriate acids as inorganic acids, for example, hydrohalic acids, e.g. hydrochloric, hydrobromic and the like; sulfuric acid; nitric acid; phosphoric acid and the like; or organic acids, for example, acetic, propanoic, hydroxy-acetic, 2-hydroxypropanoic, 2-oxopropanoic, oxalic, malonic, succinic, maleic, fumaric, malic, tartaric, 2-hydroxy-1,2,3-propanetricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic and the like acids. Conversely the salt form can be converted by treatment with alkali into the free base form.

The compounds of formula (I) containing acidic protons may be converted into their therapeutically active non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline, the benzathine, N-methyl-D-glucamine, 2-amino-2-(hydroxymethyl)-1,3-propanediol, hydrabamine salts, and salts with amino acids such as, for example, arginine, lysine and the like. Conversely the salt form can be converted by treatment with acid into the free acid form.

The term addition salt also comprises the hydrates and solvent addition forms which the compounds of formula (I) are able to form. Examples of such forms are e.g. hydrates, alcoholates and the like.

The term “quaternary amine” as used hereinbefore defines the quaternary ammonium salts which the compounds of formula (I) are able to form by reaction between a basic nitrogen of a compound of formula (I) and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate. The counterion of choice can be introduced using ion exchange resins.

The N-oxide forms of the present compounds are meant to comprise the compounds of formula (I) wherein one or several tertiary nitrogen atoms are oxidized to the so-called N-oxide.

It will be appreciated that some of the compounds of formula (I) and their N-oxides, addition salts, quaternary amines and stereochemically isomeric forms may contain one or more centers of chirality and exist as stereochemically isomeric forms.

The term “stereochemically isomeric forms” as used hereinbefore defines all the possible stereoisomeric forms which the compounds of formula (I), and their N-oxides, addition salts, quaternary amines or physiologically functional derivatives may possess. Unless otherwise mentioned or indicated, the chemical designation of compounds denotes the mixture of all possible stereochemically isomeric forms, said mixtures containing all diastereomers and enantiomers of the basic molecular structure as well as each of the individual isomeric forms of formula (I) and their N-oxides, salts, solvates or quaternary amines substantially free, i.e. associated with less than 10%, preferably less than 5%, in particular less than 2% and most preferably less than 1% of the other isomers. Thus, when a compound of formula (I) is for instance specified as (E), this means that the compound is substantially free of the (Z) isomer.

In particular, stereogenic centers may have the R- or S-configuration; substituents on bivalent cyclic (partially) saturated radicals may have either the cis- or trans-configuration. Compounds encompassing double bonds can have an E (entgegen) or Z (zusammen)-stereochemistry at said double bond. The terms cis, trans, R, S, E and Z are well known to a person skilled in the art.

Stereochemically isomeric forms of the compounds of formula (I) are obviously intended to be embraced within the scope of this invention.

Some of the compounds of formula (I) may also exist in their tautomeric form. Such forms although not explicitly indicated in the above formula (I) are intended to be included within the scope of the present invention. For instance, it is intended that formula (I) includes the tautomeric form of 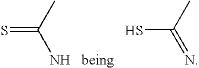
Thus, the compounds of the present invention include compounds of formula

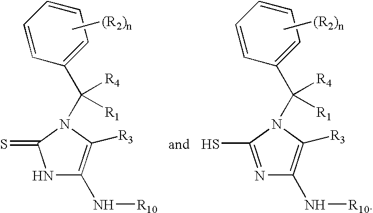

Whenever used hereinafter, the term “compounds of formula (I)” or any subgroup thereof, e.g. the compounds of formula (I′) or (I″), is meant to also include their N-oxide forms, their addition salts, their quaternary amines or their stereochemically isomeric forms. Of special interest are those compounds of formula (I) which are stereochemically pure.

Whenever used hereinbefore or hereinafter that substituents can be selected each independently out of a list of numerous definitions, such as for example for R₂, all possible combinations are intended which are chemically possible.

A first interesting embodiment of the present invention are those compounds of formula (I) wherein the carbon atom carrying the R₁ and R₄ substituent has the (S) configuration, i.e. a compound of formula (I′), or wherein the carbon atom carrying the R₁ and R₄ substituent has the (R) configuration, i.e. a compound of formula (I″), in particularly the compound of formula (I) is a compound of formula (I′).

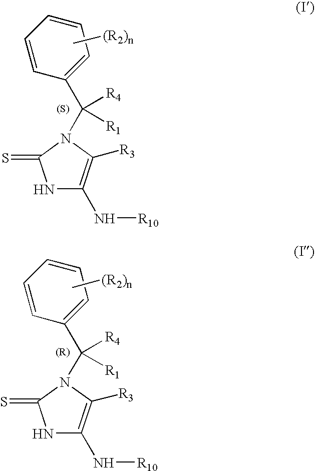

A second interesting embodiment of the present invention are those compounds of formula (I) or any subgroup thereof as mentioned hereinbefore as interesting embodiment wherein
R₁ represents hydrogen, C₁₋₆alkyl, C₃₋₇cycloalkyl, C₁₋₆alkyloxyC₁₋₆alkyl, di(C₁₋₆alkyl)aminoC₁₋₆alkyl, aryl or heteroaryl;
each R₂ independently represents halo, C₁₋₆alkyl, C₁₋₆alkyloxy, C₁₋₆alkylthio, polyhaloC₁₋₆alkyl, polyhaloC₁₋₆alkyloxy, cyano, aminocarbonyl, amino, mono- or di(C₁₋₄alkyl)amino, nitro, aryl or aryloxy;
R₃ represents cyano, C(═O)—O—R₅, C(═O)—NR₆ₐR_{6b} or C(═O)—R₇; or a cyclic ring system selected from 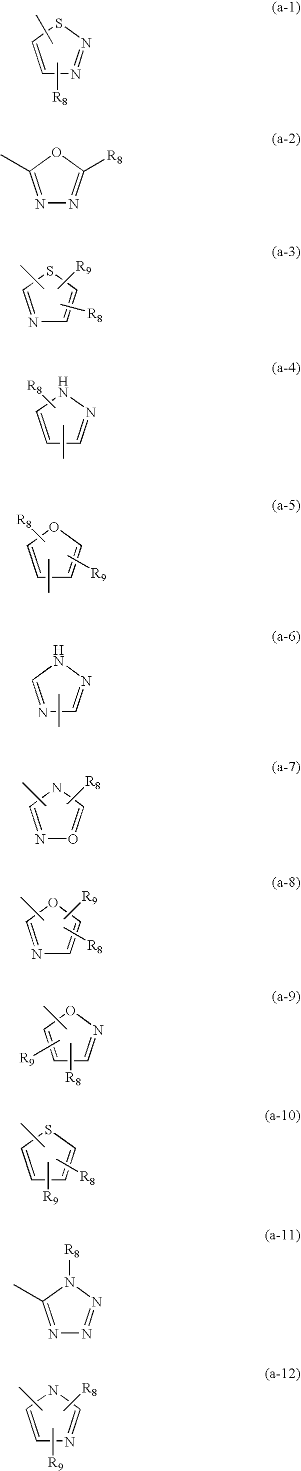 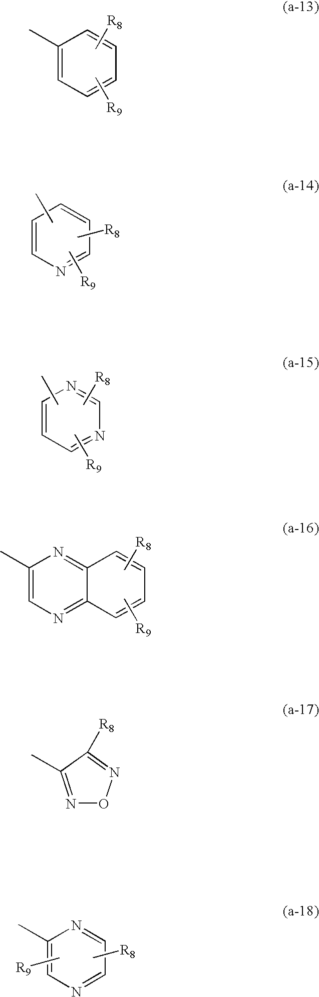
R₄ represents hydrogen or C₁₋₆alkyl;
R₅ represents hydrogen, C₁₋₆alkyl, hydroxyC₁₋₆alkyl, C₂₋₆alkenyl, C₂₋₆alkynyl, polyhaloC₁₋₆alkyl, C₁₋₆alkyloxyC₁₋₆alkyl, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl, aryl or arylC₁₋₆alkyl;
R₆ₐ and R_{6b} each independently represent hydrogen, C₁₋₆alkyl, amino, mono- or di(C₁₋₄alkyl)amino, arylNH—, aminoC₁₋₆alkyl, mono- or di(C₁₋₄alkyl)amino-C₁₋₆alkyl, C₁₋₆alkylcarbonylamino, aminocarbonylamino, C₁₋₆alkyloxy, carbonylamino or hydroxyC₁₋₆alkyl; or
R₆ₐ and R_{6b} taken together with the nitrogen to which they are attached form pyrrolidinyl, imidazolidinyl, pyrazolidinyl, piperidinyl, piperazinyl, morpholinyl, thiomorpholinyl or piperazinyl substituted with C₁₋₆alkyl;
R₇ represents hydrogen, C₁₋₆alkyl, hydroxyC₁₋₆alkyl, C₂₋₆alkenyl, C₂₋₆alkynyl, polyhaloC₁₋₆alkyl, C₁₋₆alkyloxyC₁₋₆alkyl, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl, aryl or heteroaryl; each R₈ independently represents hydrogen, halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, hydroxyC₁₋₆alkylamino, aryl, aryloxy, piperidinyl, piperidinylamino, morpholinyl, piperazinyl or nitro;
each R₉ independently represents hydrogen, halo or C₁₋₆alkyl;
R₁₀ represents hydrogen, C₁₋₆alkyl, C₁₋₆alkylcarbonyl, arylcarbonyl, heteroarylcarbonyl, —C(═O)—NH—R₅, —C(═S)—NH—R₅ or —S(═O)₂—R₅;
n is 1, 2, 3, 4 or 5;
aryl represents phenyl or phenyl substituted with one, two, three, four or five substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, phenyloxy or nitro;
heteroaryl represents furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, each of said heterocycles optionally being substituted with one or two substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, or nitro.

A third interesting embodiment of the present invention are those compounds of formula (I), (I′) or (I″) or any subgroup thereof as mentioned hereinbefore as interesting embodiment wherein R₃ represents cyano, C(═O)—O—R₅, C(═O)—NR₆ₐR_{6b}, or a cyclic ring system as defined hereinabove, in particular wherein R₃ represents C(═O)—O—R₅, more in particular C(═O)—O—C₁₋₆alkyl, e.g. methoxycarbonyl.

A fourth interesting embodiment of the present invention are those compounds of formula (I), (I′) or (I″) or any subgroup thereof as mentioned hereinbefore as interesting embodiment wherein R₁₀ represents hydrogen, C₁₋₆alkylcarbonyl, C₁₋₆alkyloxycarbonyl, arylcarbonyl, heteroarylcarbonyl, —C(═O)—NH—R₅, —C(═S)—NH—R₅ or —S(═O)₂—R₅, in particular hydrogen, C₁₋₆alkylcarbonyl, C₁₋₆alkyloxycarbonyl, arylcarbonyl, heteroarylcarbonyl, or —C(═O)—NH—R₅; even more in particular C₁₋₆alkylcarbonyl, C₁₋₆alkyloxycarbonyl, arylcarbonyl, heteroarylcarbonyl, or —C(═O)—NH—R₅; or R₁₀ represents hydrogen, C₁₋₆alkylcarbonyl, arylcarbonyl, heteroarylcarbonyl, —C(═O)—NH—R₅, —C(═S)—NH—R₅ or —S(═O)₂—R₅, in particular C₁₋₆alkylcarbonyl, arylcarbonyl, heteroarylcarbonyl, —C(═O)—NH—R₅, —C(═S)—NH—R₅ or —S(═O)₂—R₅, more in particular C₁₋₆alkylcarbonyl, arylcarbonyl, heteroarylcarbonyl or —C(═O)—NH—R₅, even more in particular C₁₋₆alkylcarbonyl, arylcarbonyl or heteroarylcarbonyl.

A fifth interesting embodiment of the present invention are those compounds of formula (I), (I′) or (I″) or any subgroup thereof as mentioned hereinbefore as interesting embodiment wherein n is 2 or 3, in particular n is 2.

A sixth interesting embodiment of the present invention are those compounds of formula (I), (I′) or (I″) or any subgroup thereof as mentioned hereinbefore as interesting embodiment wherein n is 2 and said two substituents are placed in meta and para position.

A seventh interesting embodiment of the present invention are those compounds of formula (I), (I′) or (I″) or any subgroup thereof as mentioned hereinbefore as interesting embodiment wherein R₃ represents a radical of formula (a-1), (a-2), (a-3), (a-4), (a-5), (a-6), (a-7), (a-9), (a-10), (a-11), (a-12), (a-13), (a-14), (a-15), (a-16) or (a-18); preferably a radical of formula (a-2), (a-3), (a-4), (a-5), (a-6), (a-7), (a-11), (a-12), (a-13), (a-14) or (a-15); more preferably a radical of formula (a-2), (a-3), (a-5), (a-6), (a-7), (a-12), (a-13), (a-14) or (a-15), in particular wherein R₃ represents a radical of formula (a-2) or (a-15).

An eight interesting embodiment of the present invention are those compounds of formula (I), (I′) or (I″) or any subgroup thereof as mentioned hereinbefore as interesting embodiment wherein R₂ represents halo, C₁₋₆alkyl, C₁₋₆alkyloxy or polyhaloC₁₋₆alkyl, in particular halo or polyhaloC₁₋₆alkyl, more in particular halo, e.g. chloro, fluoro or trifluoromethyl, preferably chloro.

A ninth interesting embodiment of the present invention are those compounds of formula (I), (I′) or (I″) or any subgroup thereof as mentioned hereinbefore as interesting embodiment wherein R₁ is hydrogen, methyl, ethyl, n-propyl, methoxymethyl, cyclohexyl, cyclopropyl, dimethylaminomethyl, 2-thienyl, 3,4-dichlorophenyl; preferably R₁ is C₁₋₆alkyl or

C₁₋₆alkyloxyC₁₋₆alkyl, in particular methyl, ethyl, n-propyl, methoxymethyl, more preferably R₁ is C₁₋₆alkyl, in particular methyl, ethyl and propyl, more in particular methyl, ethyl or n-propyl; most preferred R₁ is ethyl.

A tenth interesting embodiment of the present invention are those compounds of formula (I), (I′) or (I″) or any subgroup thereof as mentioned hereinbefore as interesting embodiment wherein R₄ represents hydrogen.

An eleventh interesting embodiment of the present invention are those compounds of formula (I), (I′) or (I″) or any subgroup thereof as mentioned hereinbefore as interesting embodiment wherein R₅ represents C₁₋₆alkyl, arylC₁₋₆alkyl or C₁₋₆alkyloxyC₁₋₆alkyl optionally substituted with C₁₋₆alkyloxy; in particular C₁₋₆alkyl, arylC₁₋₆alkyl or C₁₋₆alkyloxyC₁₋₆alkyl.

A twelfth interesting embodiment of the present invention are those compounds of formula (I), (I′) or (I″) or any subgroup thereof as mentioned hereinbefore as interesting embodiment which are stereochemically pure.

A thirteenth interesting embodiment of the present invention are those compounds of formula (I), (I′) or (I″) wherein one or more, preferably all of the following restrictions apply:
a) R₁ represents C₁₋₆alkyl; in particular ethyl;
b) R₂ represents halo, polyhaloC₁₋₆alkyl or aryloxy; in particular halo, e.g. chloro and fluoro; more in particular chloro;
c) R₁₀ represents hydrogen, C₁₋₆alkylcarbonyl, C₁₋₆alkyloxycarbonyl, —C(═O)—NH—R₅, arylcarbonyl, or heteroarylcarbonyl; in particular hydrogen, C₁₋₆alkylcarbonyl, arylcarbonyl, or heteroarylcarbonyl, more in particular hydrogen, C₁₋₆alkylcarbonyl, phenylcarbonyl or heteroarylcarbonyl; wherein heteroaryl represents pyrrolidinyl, tetrahydrofuranyl, piperazinyl optionally substituted with C₁₋₆alkyl or arylC₁₋₆alkyl, piperidinyl, morpholinyl, pyrazinyl, pyridyl, isoxazolyl, oxadiazolyl, pyrimidinyl or furanyl; more in particular wherein heteroaryl represents pyrazinyl, pyridyl, isoxazolyl, oxadiazolyl, pyrimidinyl or furanyl;
d) R₃ represents cyano, C(═O)—O—R₅, C(═O)—NR₆ₐR_{6b} or C(═O)—R₇; in particular C(═O)—O—R₅;
e) R₄ represents hydrogen;
f) n is 2 or 3; preferably n is 2.

A fourteenth interesting embodiment of the present invention are those compounds of formula (I), (I′) or (I″) wherein one or more of the following restrictions apply:
a) R₁ represents C₁₋₆alkyl, in particular ethyl;
b) R₂ represents halo; in particular chloro;
c) R₃ represents C(═O)—O—R₅; in particular C(═O)—O—C₁₋₆alkyl; more in particular methoxycarbonyl;
d) R₁₀ represents hydrogen, C₁₋₆alkylcarbonyl, C₁₋₆alkyloxycarbonyl, —C(═O)—NH—R₅, arylcarbonyl or heteroarylcarbonyl; in particular hydrogen, C₁₋₆alkylcarbonyl, arylcarbonyl or heteroarylcarbonyl; more in particular hydrogen, methylcarbonyl, pyrazinylcarbonyl, furanylcarbonyl or pyridylcarbonyl;
e) R₄ represents hydrogen;
f) n is 2.

A fifteenth interesting embodiment of the present invention are those compounds of formula (I), (I′) or (I″) wherein one or more of the following restrictions apply:
a) R₁ represents C₁₋₆alkyl; in particular ethyl;
b) R₂ represents halo; in particular chloro;
c) R₃ represents C(═O)—O—R₅; in particular C(═O)—O—C₁₋₆alkyl; more in particular methoxycarbonyl;
d) R₁₀ represents hydrogen, C₁₋₆alkylcarbonyl, C₁₋₆alkyloxycarbonyl or heteroarylcarbonyl; e.g. hydrogen, methylcarbonyl, methyloxycarbonyl, tetrahydrofuranylcarbonyl, morpholinylcarbonyl, pyrazinylcarbonyl, furanylcarbonyl or pyridylcarbonyl; in particular R₁₀ represents hydrogen, C₁₋₆alkylcarbonyl or heteroarylcarbonyl; e.g. hydrogen, methylcarbonyl, pyrazinylcarbonyl, furanylcarbonyl or pyridylcarbonyl;
e) R₄ represents hydrogen;
f) n is 2.

In general, compounds of formula (I) wherein R₁₀ represents hydrogen, said compounds being represented by formula (I-a), can be prepared by reacting an intermediate of formula (II) with phosphorazidic acid diphenyl ester in the presence of a suitable base, such as for example N,N-diethylethanamine, and a suitable solvent, such as for example an alcohol, e.g. tert-butanol.

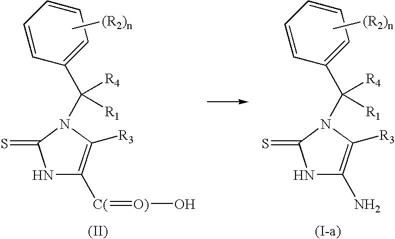

Compounds of formula (I-a) can be converted into a compound of formula (I) wherein R₁₀ represents C₁₋₆alkylcarbonyl, said compound being represented by formula (I-b), by reaction with an intermediate of formula (III).

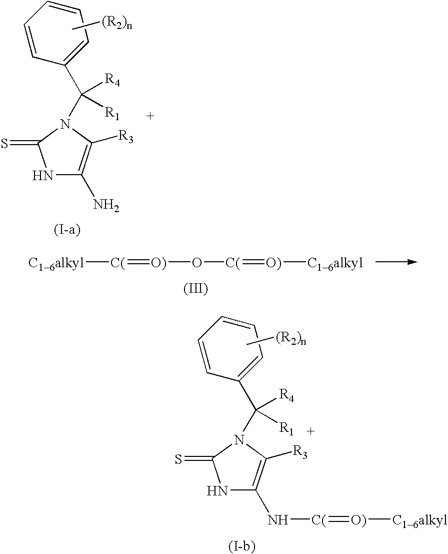

Compounds of formula (I-a) can be converted into a compound of formula (I) wherein R₁₀ represents C₁₋₆alkylcarbonyl, C₁₋₆alkyloxycarbonyl arylcarbonyl or heterocarbonyl, said R₁₀ being represented by R₁₀ₐ and said compound being represented by formula (I-c), by reaction with an intermediate of formula (IV) in the presence of suitable coupling agent such as for example N′-(ethylcarbonimidoyl)-N,N-dimethyl-1,3-propanediamine optionally together with 1-hydroxy-1H-benzotriazole, a suitable solvent, such as for example N,N-dimethylformamide, and a suitable base, such as for example N,N-dimethyl-4-pyridinamine.

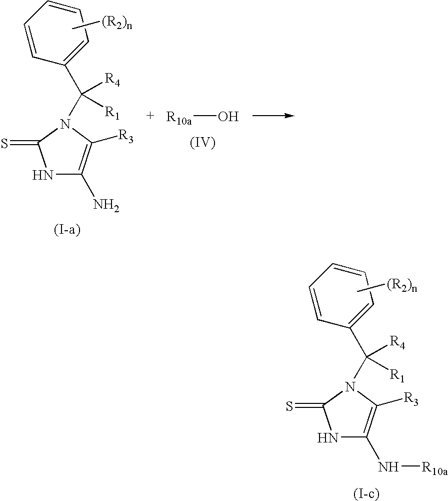

Compounds of formula (I-a) can also be converted into a compound of formula (I-c) by reaction with an intermediate of formula (V) wherein W₁ represents a suitable leaving group, such as for example halo, e.g. chloro and the like, in the presence of a suitable base, such as for example N,N-diethylethanamine, and a suitable solvent, such as for example methylene chloride.

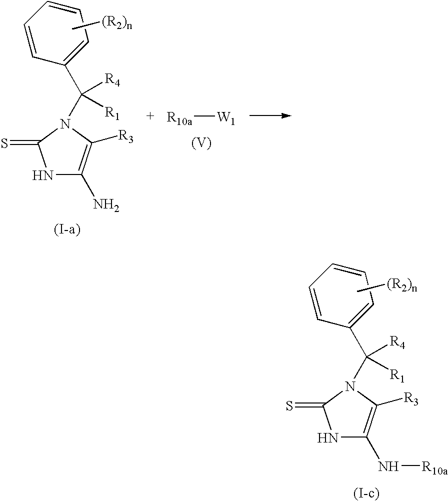

Compounds of formula (I-a) can also be converted into a compound of formula (I-c) wherein R₁₀ₐ represents imidazol-1-ylcarbonyl, said compound being represented by formula (I-c-1), by reaction with 1,1′-carbonyldiimidazole in the presence of a suitable solvent, such as for example CH₂Cl₂.

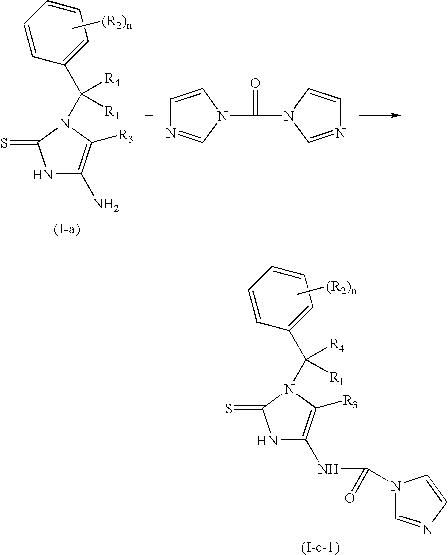

Compounds of formula (I-c-1) can be converted into a compound of formula (I-c) wherein R₁₀ₐ represents morpholinylcarbonyl, said compound being represented by formula (I-c-2), by reaction with morpholine in the presence of a suitable solvent, such as for example tetrahydrofuran.

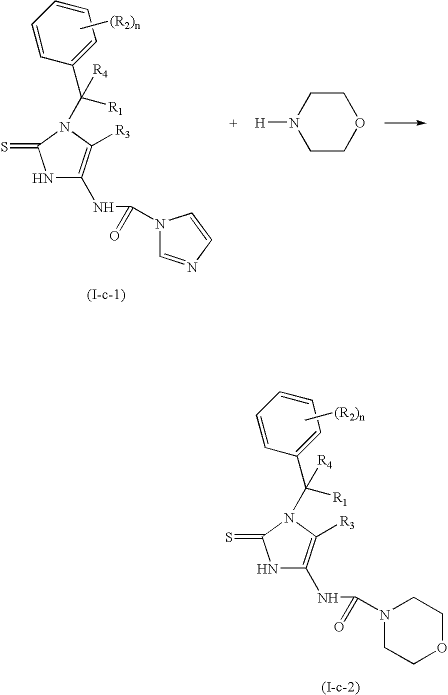

Compounds of formula (I-c-1) can also be converted into a compound of formula (I-c) wherein R₁₀ represents C₁₋₆alkyloxycarbonyl, said compound being represented by formula (I-c-3), by reaction with C₁₋₆alkyl-OH.

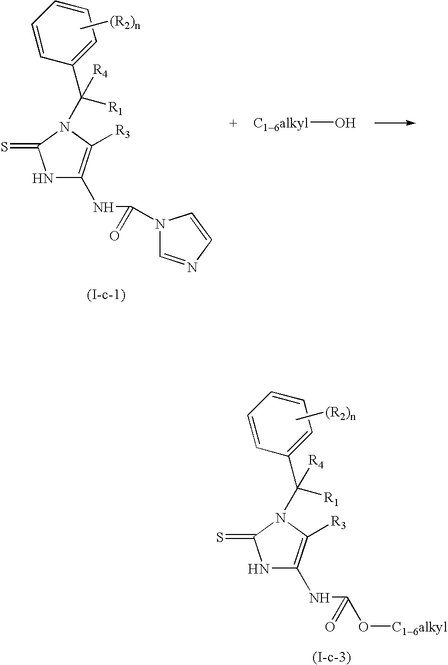

Compounds of formula (I) wherein R₁₀ represents —C(═O)—NHR₅ respectively —C(═S)—NH—R₅ wherein R₅ is other than hydrogen, said R₅ being represented by R₅ₐ and said compounds being represented by formula (I-d) respectively (I-e), can be prepared by reacting a compound of formula (I-a) with R₅ₐ—N═C═O respectively R₅ₐ—N═C═S in the presence of a suitable solvent, such as for example tetrahydrofuran, methylene chloride, dioxane, and optionally in the presence of a suitable base, such as for example N,N-diethylethanamine.

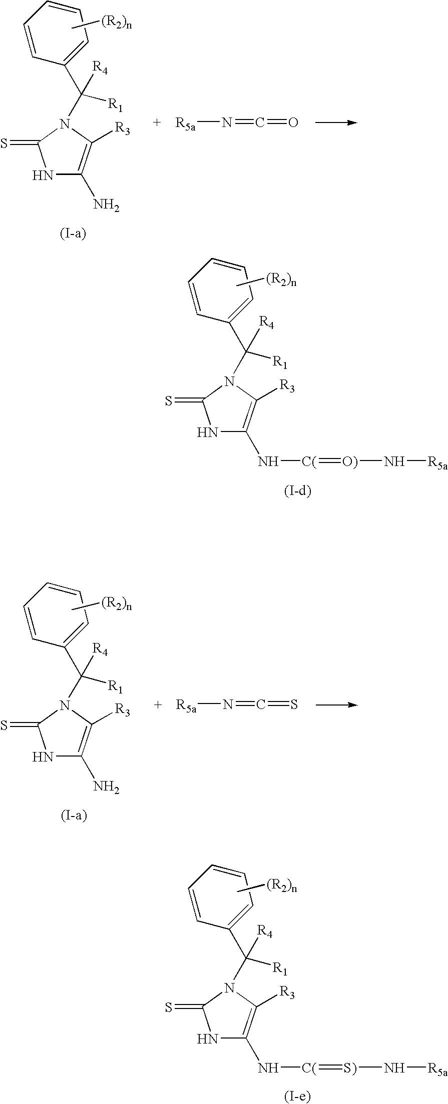

Compounds of formula (I-d) wherein R₅ₐ represents arylC₁₋₆alkyl or C₁₋₆alkyloxyC₁₋₆alkyl optionally substituted with C₁₋₆alkyloxy, said R₅ₐ being represented by R_{5b} and said compounds being represented by formula (I-d-1), may also be prepared by reacting a compound of formula (I-c-1) with NH₂—R_{5b}, in the presence of a suitable solvent, such as for example tetrahydrofuran.

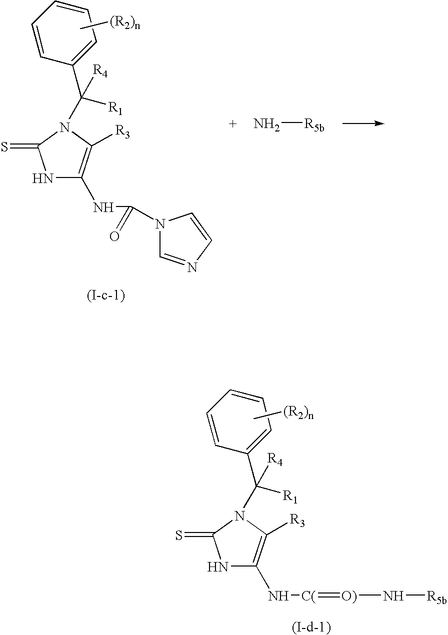

Compounds of formula (I) wherein R₁₀ represents —C(═O)—NH₂ respectively —C(═S)—NH₂, said compounds being represented by formula (I-f) respectively (I-g), can be prepared by reacting a compound of formula (I-a) with W₅—N═C═O respectively W₅—N═C═S wherein W₅ represents a suitable leaving group, such as for example —Si(CH₃)₃ or —S(═O)₂—Cl, in the presence of a suitable solvent, such as for example tetrahydrofuran, methylene chloride, dioxane, and optionally in the presence of a suitable base, such as for example N,N-diethylethanamine, followed by removing the leaving group with a suitable acid, such as for example hydrochloric acid and the like.

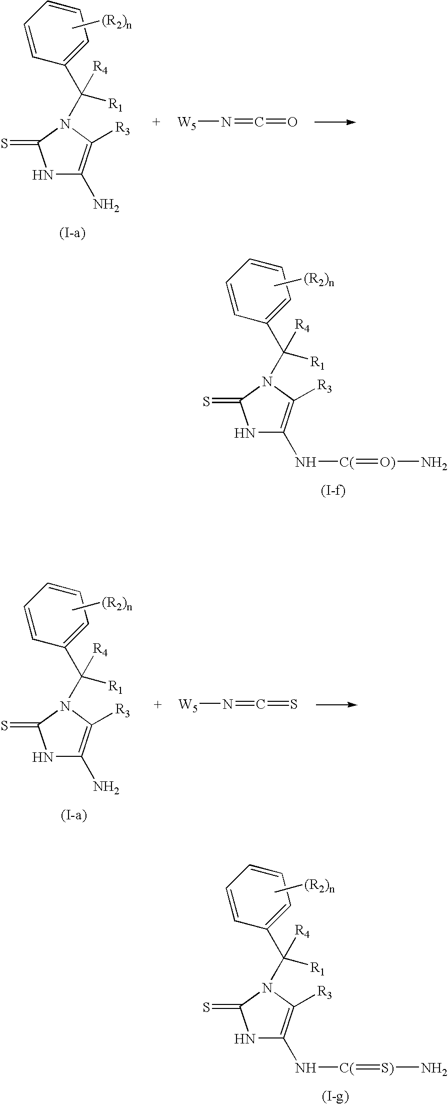

Compounds of formula (I) wherein R₁₀ represents —S(═O)₂—R₅, said compounds being represented by formula (I-h), can be prepared by reacting a compound of formula (I-a) with an intermediate of formula W₂—S(═O)₂—R₅ wherein W₂ represents a suitable leaving group, such as for example halo, e.g. chloro and the like, in the presence of a suitable base, such as for example N,N-diethylethanamine, and a suitable solvent, such as for example tetrahydrofuran, N,N-dimethylformamide.

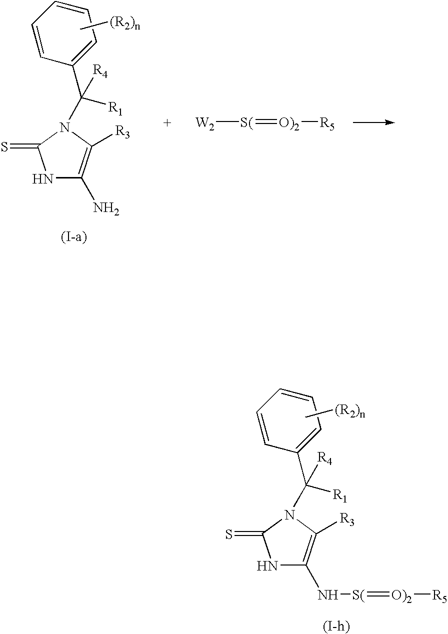

Compounds of formula (I′) can be prepared according to the above described reactions but starting from an intermediate wherein the carbon atom carrying the R₁ and R₄ substituent has the (S) configuration.

Alternatively, compounds of formula (I) wherein the carbon atom carrying the R₁ and R₄ substituent has the (R) configuration can be prepared according to the above described reactions but starting from an intermediate wherein the carbon atom carrying the R₁ and R₄ substituent has the (R) configuration.

The compounds of formula (I) may further be prepared by converting compounds of formula (I) into each other according to art-known group transformation reactions.

The compounds of formula (I) may be converted to the corresponding N-oxide forms following art-known procedures for converting a trivalent nitrogen into its N-oxide form. Said N-oxidation reaction may generally be carried out by reacting the starting material of formula (I) with an appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chlorobenzenecarboperoxoic acid, peroxoalkanoic acids, e.g. peroxoacetic acid, alkylhydroperoxides, e.g. tert.butyl hydro-peroxide. Suitable solvents are, for example, water, lower alcohols, e.g. ethanol and the like, hydrocarbons, e.g. toluene, ketones, e.g. 2-butanone, halogenated hydrocarbons, e.g. dichloromethane, and mixtures of such solvents.

Compounds of formula (I) wherein R₃ represents C(═O)—O—C₁₋₆alkyl, can also be converted into a compound of formula (I) wherein R₃ represents C(═O)—NR₆ₐR_{6b}, by reaction with the appropriate base of formula NHR₆ₐR_{6b} in a suitable solvent, such as for example H₂O.

Compounds of formula (I) wherein R₃ represents C(═O)—O—C₁₋₆alkyl, can also be converted into a compound of formula (I) wherein R₃ represents C(═O)—O—C₁₋₆alkyl-O—C₁₋₆alkyl, by reaction with HO—C₁₋₆alkyl-O—C₁₋₆alkyl in the presence of NaBH₄.

Compounds of formula (I) wherein R₃ represents C(═O)—NR₆ₐR_{6b} can be converted into a compound of formula (I) wherein R₃ represents C(═O)—C₁₋₆alkyl by reaction with chloroC₁₋₆alkyMg in a suitable solvent such as tetrahydrofuran.

Compounds of formula (I) wherein R₃ represents cyano, can be converted into a compound of formula (I) wherein R₃ represents aminocarbonyl by hydrolysis with a suitable acid, such as for example sulfuric acid.

Some of the compounds of formula (I) and some of the intermediates in the present invention may contain an asymmetric carbon atom. Pure stereochemically isomeric forms of said compounds and said intermediates can be obtained by the application of art-known procedures. For example, diastereoisomers can be separated by physical methods such as selective crystallization or chromatographic techniques, e.g. counter current distribution, liquid chromatography and the like methods. Enantiomers can be obtained from racemic mixtures by first converting said racemic mixtures with suitable resolving agents such as, for example, chiral acids, to mixtures of diastereomeric salts or compounds; then physically separating said mixtures of diastereomeric salts or compounds by, for example, selective crystallization or chromatographic techniques, e.g. liquid chromatography and the like methods; and finally converting said separated diastereomeric salts or compounds into the corresponding enantiomers. Pure stereochemically isomeric forms may also be obtained from the pure stereochemically isomeric forms of the appropriate intermediates and starting materials, provided that the intervening reactions occur stereospecifically.

An alternative manner of separating the enantiomeric forms of the compounds of formula (I) and intermediates involves liquid chromatography, in particular liquid chromatography using a chiral stationary phase.

Some of the intermediates and starting materials are known compounds and may be commercially available or may be prepared according to art-known procedures.

Intermediates of formula (II) can be prepared by reacting an intermediate of formula (VI) with an intermediate of formula (VII) in the presence of a suitable base, such as for example NaOCH₃ or NaOC(CH₃)₃, followed by reaction with KSCN and a suitable acid, such as for example hydrochloric acid (36%) and the like, in the presence of a suitable solvent, such as for example tetrahydrofuran.

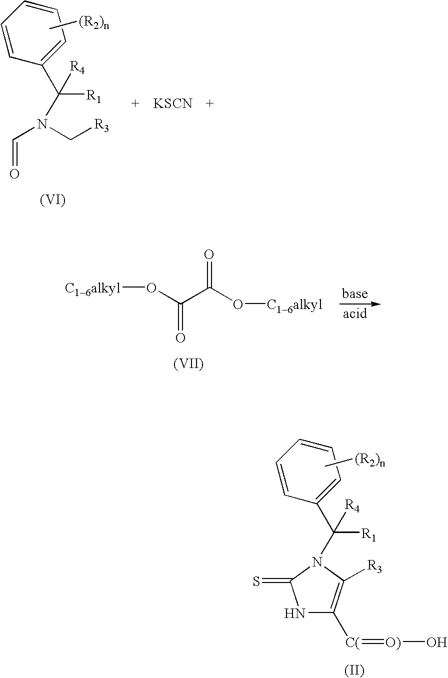

Intermediates of formula (JT) can also be prepared by reacting an intermediate of formula (VIII) with a suitable base, such as NaOH, in the presence of a suitable solvent, such as for example H₂O, tetrahydrofuran or an appropriate alcohol, e.g. methanol and the like, or by reaction with a suitable acid, such as for example CF₃CH₂COOH in the presence of a suitable solvent, such as for example methylene chloride.

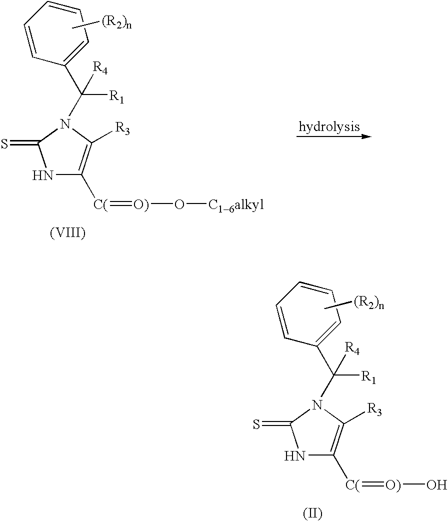

Intermediates of formula (VI) can be prepared by reacting an intermediate of formula (IX) with a H—C(═O)— introducing agent, such as for example formic acid or n-butyl formate, in the presence of a suitable solvent, such as for example xylene.

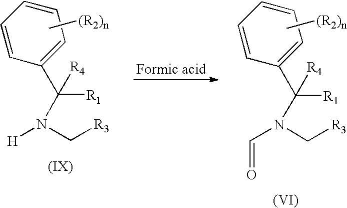

The above reaction may result in stereochemically pure intermediates of formula (VI) when starting from stereochemically pure intermediates of formula (IX).

Intermediates of formula (IX) can be prepared by reacting an intermediate of formula (X) with an intermediate of formula (XI) wherein W₃ represents a suitable leaving group, such as for example a halogen, e.g. bromo, chloro and the like, in the presence of a suitable base, such as for example N,N-diethylethanamine or N,N-diisopropyl-ethanamine, and a suitable solvent, such as for example N,N-dimethylformamide.

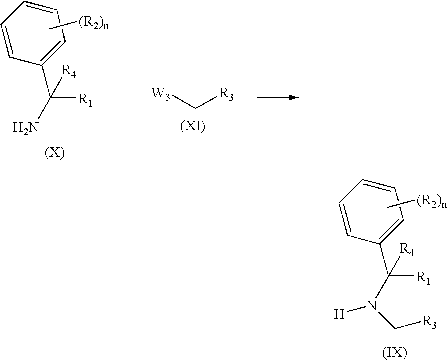

The above reaction may result in stereochemically pure intermediates of formula (IX) when starting from stereochemically pure intermediates of formula (X).

Intermediates of formula (X) wherein R₄ represents hydrogen, said intermediates being represented by formula (X-a), can be prepared by reducing an intermediate of formula (XII) in the presence of a suitable reducing agent, such as H₂, a suitable catalyst, such as for example Raney Nickel, a suitable catalyst poison, such as for example a thiophene solution, and a suitable solvent, such as for example an alcohol, e.g. methanol, in the presence of a suitable base, e.g. NH₃. Alternatively, said reaction can also be performed in the presence of Zn and a suitable acid, such as for example acetic acid.

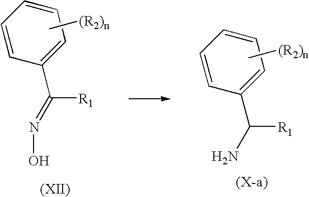

Intermediates of formula (XII) can be prepared by reacting an intermediate of formula (XIII) with NH₂—OH in the presence of a suitable base, such as for example NaOC(═O)CH₃ or Na₂CO₃, and a suitable solvent, such as for example an alcohol, e.g. methanol.

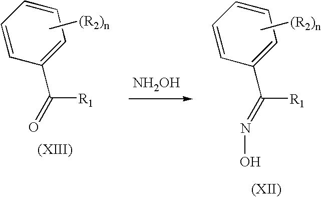

Alternatively to the methods described above, intermediates of formula (X) can also be prepared from an azido derivative of formula (XIV) by reaction with triphenylphosphine in the presence of a suitable solvent, such as for example tetrahydrofuran and H₂O.

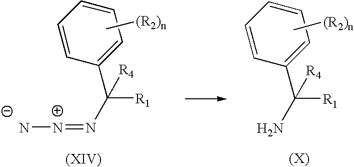

Intermediates of formula (X) can also be prepared from an intermediate of formula (XIV) by catalytic hydrogenation in the presence of H₂, a suitable catalyst, such as for example Pt/C (5%), and a suitable solvent, such as for example an alcohol, e.g. methanol.

Intermediates of formula (XIV) can be prepared by reacting an intermediate of formula (XV) with phosphorazidic acid diphenylester in the presence of 2,3,4,6,7,8,9,10-octahydropyrimido[1,2-a]azepine and a suitable solvent, such as for example toluene.

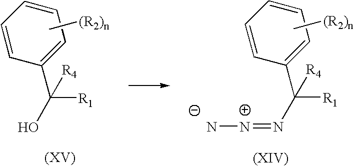

Intermediates of formula (XV) wherein R₁ is C₁₋₆alkyl and wherein R₄ is hydrogen, said intermediates being represented by formula (XV-a), can be prepared by reacting an intermediate of formula (XIII) wherein R₁ represents hydrogen, said intermediates being represented by formula (XIII-a), with (C₁₋₆alkyl)₂Zn, N,N′-1,2-cyclohexanediylbis[1,1,1-trifluoro]methanesulfonamide, Ti(i-PrO)₄ and toluene.

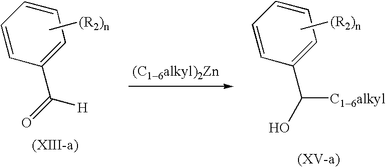

Intermediates of formula (X) can be prepared as described hereinabove.

The intermediates of formula (X) may contain a chiral center at the carbon atom carrying the R₁ and R₄ substituent depending on the substituents representing R₁ and R₄. In case said carbon atom represents a chiral center, stereospecific intermediates of formula (X) represented by formula (X-b), can be prepared by reacting a stereospecific intermediate of formula (XIV), represented by formula (XIV-a), with triphenylphosphine, in the presence of a suitable solvent, such as for example tetrahydrofuran and water. 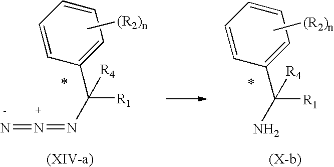
* indicates the chiral center and may be (R) or (S) depending on the R₁ and R₄ substituents

When a stereospecific intermediate of formula (X-b) is reacted further according to the methods described hereinabove, the resulting intermediates are also stereospecific and finally the resulting final compounds are also stereospecific.

Intermediates of formula (XIV-a) can be prepared by reacting a stereospecific intermediate of formula (XV) represented by formula (XV-a) with phosphorazidic acid diphenyl ester in the presence of 2,3,4,6,7,8,9,10-octahydro-pyrimido[1,2-a]azepine and in the presence of a suitable solvent, such as for example toluene. 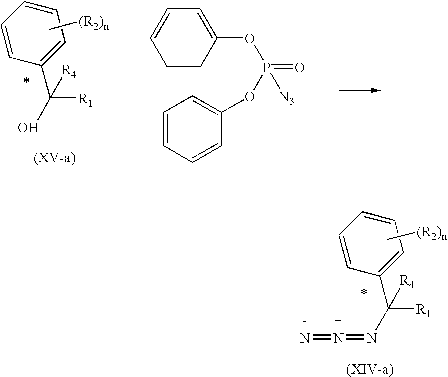
* indicates the chiral center and may be (R) or (S) depending on the R₁ and R₄ substituents

Stereospecific intermediates of formula (XV-a) wherein R₄ is hydrogen and R₁ is methyl, ethyl, or n-propyl, said R₁ being represented by Alk and said intermediates being represented by formula (XV-a-1) and (XV-a-2), can be prepared by reacting an intermediate of formula (XVI) with ZnAlk₂ wherein Alk represents methyl, ethyl or n-propyl, in the presence of a stereospecific catalyst, such as for example N,N′-(1R,2R)-1,2-cyclohexanediylbis[1,1,1-trifluoro]-methanesulfonamide respectively N,N′-(1S,2S)-1,2-cyclohexanediylbis[1,1,1-trifluoro]-methanesulfonamide, Ti(iPrO)₄ and a suitable solvent, such as for example toluene.

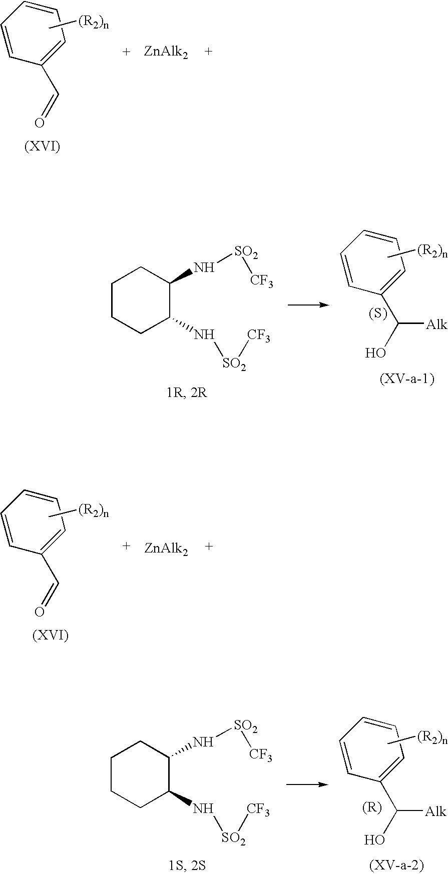

Intermediates of formula (VIII) can be prepared by reacting an intermediate of formula (VI) with an intermediate of formula (VII) in the presence of a suitable base, such as for example NaOCH₃ or NaOC(CH₃)₃ and the like, and KSCN in the presence of a suitable solvent, such as for example tetrahydrofuran.

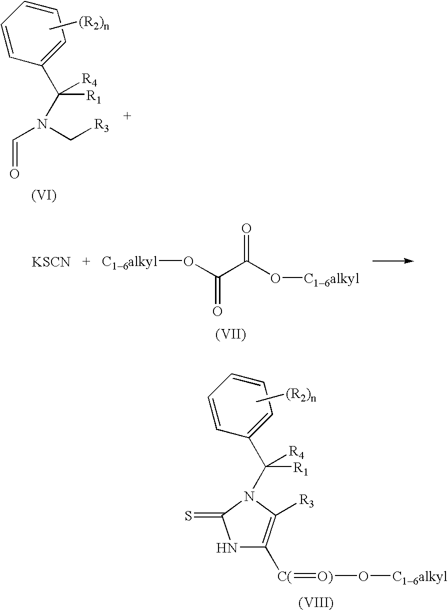

Intermediates of formula (VIII) can also be prepared by reacting an intermediate of formula (XVII) with an appropriate acid, such as hydrochloric acid or acetic acid, optionally in the presence of a suitable solvent, such as for example 1,4-dioxane.

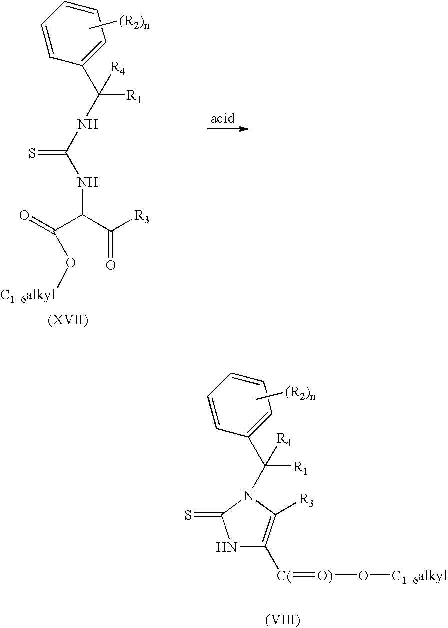

Intermediates of formula (XVII) can be prepared by reacting an intermediate of formula (XVIII) with an intermediate of formula (XIX) in the presence of a suitable base, such as for example dipotassium carbonate, and a suitable solvent, such as for example dioxane or tetrahydrofuran and water.

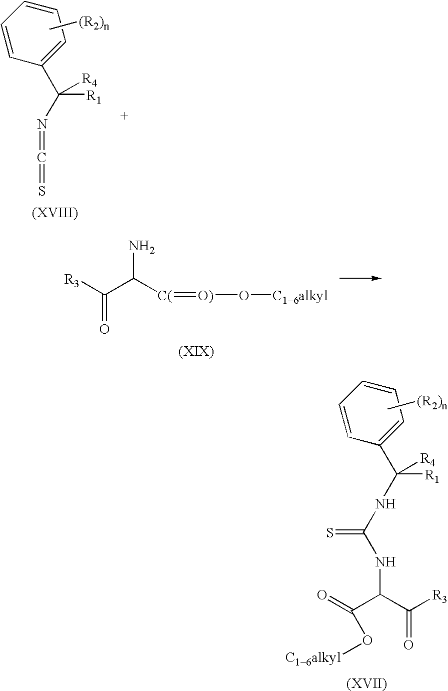

Intermediates of formula (XVIII) can be prepared by reacting an intermediate of formula (X) with C(═S)Cl₂ in the presence of a suitable base, such as for example N,N-diisopropylethanamine, and a suitable solvent, such as for example methylene chloride.

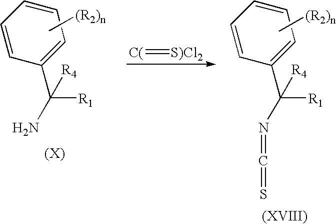

Intermediates of formula (VIII) wherein R₃ represents optionally substituted thiazolyl, said intermediates being represented by formula (VIII-a), can be prepared by reacting an intermediate of formula (XX) with a suitable acid, such as for example trifluoroacetic acid.

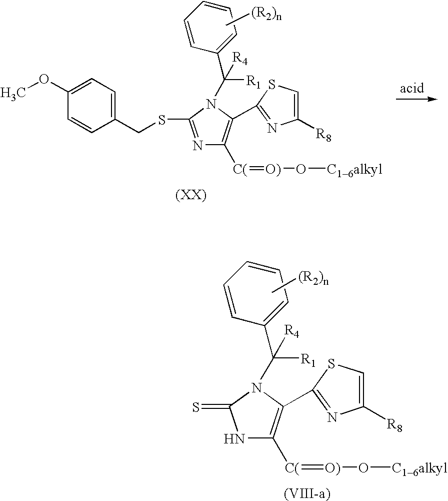

Intermediates of formula (XX) can be prepared by reacting an intermediate of formula (XXI) with an intermediate of formula (XXII) wherein W₄ represents a suitable leaving group, such as for example halo, e.g. chloro, bromo and the like, in the presence of a suitable solvent, such as for example an alcohol, e.g. ethanol.

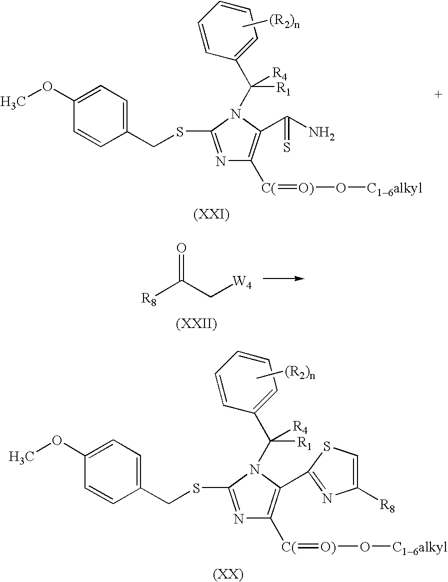

Intermediates of formula (XXI) can be prepared by reacting an intermediate of formula (XXIII) with H₂S in the presence of a suitable base, such as for example N,N-diisopropylethanamine, and a suitable solvent, such as for example pyridine.

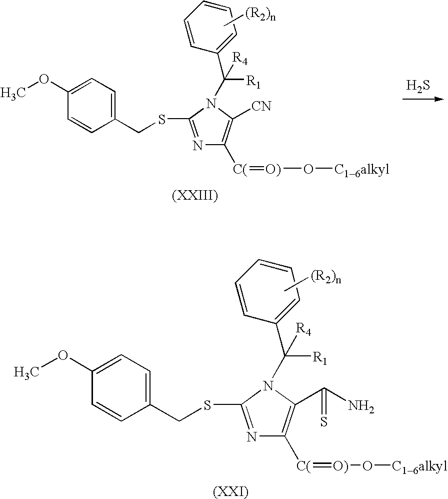

Intermediates of formula (XXIII) can be prepared by reacting an intermediate of formula (VIII) wherein R₃ represents cyano, said intermediates being represented by formula (VIII-b), with 4-methoxy-benzenemethanol in the presence of a suitable acid, such as for example trifluoroacetic acid, and a suitable solvent, such as for example methylene chloride.

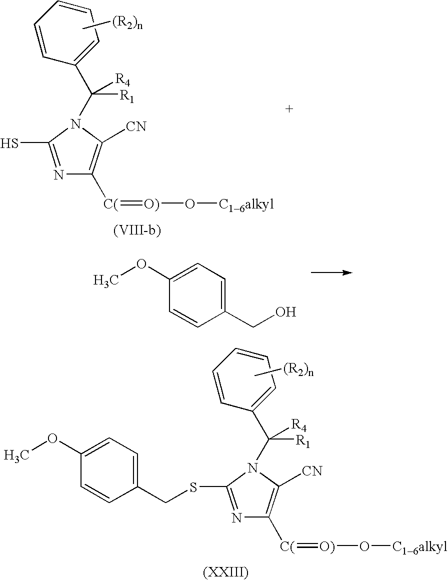

The compounds of formula (I) and any subgroup thereof, e.g. compounds of formula (I′) or (I″), show CCR2 receptor antagonistic properties.

The C—C chemokine receptor 2 (CCR2) and its ligand monocyte chemoattractant (chemotactic) protein (MCP-1; in new chemokine nomenclature also called CCL2) are recognized to be implicated in both acute and chronic inflammatory processes.

Chemokines (contraction of “chemotactic cytokines”) are most important regulators of leukocyte trafficking. This biological role is exerted by interacting—on target cells—with seven-transmembrane-domain receptors that are coupled to heterodimeric G proteins. Chemokines are mainly grouped into 2 major families (C—C or C—X—C family) dependent on the presence of an amino acid (represented by X) between the two conserved cysteine residues (represented by C) near the amino terminus. In general, chemokines from the C—C family attract monocytes, macrophages, T cells and NK cells.

A chemokine, which acts through the CCR2 receptor, is MCP-1 as indicated above. Therefore, the CCR2 receptor is also known as the MCP-1 receptor. MCP-2, MCP-3 and MCP-4 may also act, at least in part, through this receptor.

It is recognized that the CCR2 receptor and MCP-1 play a role in the pathophysiology of various inflammatory diseases. Therefore, CCR2 receptor antagonists, which block the CCR2 receptor, have potential as pharmaceutical agents to combat inflammatory conditions such as arthritis, osteoarthritis, rheumatoid arthritis, glomerulonephritis, diabetic nephropathy, lung fibrosis, idiopathic pulmonary fibrosis, sarcoidosis, vasculitis, hepatitis, nonalcoholic steatohepatitis, inflammatory conditions of the brain such as Alzheimer's disease, restenosis, alveolitis, asthma, allergic rhinitis, allergic conjunctivitis, atherosclerosis, psoriasis, delayed-type hypersensitivity reactions of the skin, inflammatory bowel disease, acute or chronic brain inflammation, e.g. multiple sclerosis, autoimmune encephalomyelitis, chronic obstructive pulmonary disease (COPD), uveitis, dermatitis, atopic dermatitis. CCR2 receptor antagonists may also be useful to treat autoimmune diseases such as diabetes or transplant rejection, stroke, reperfusion injury, ischemia, cancer, myocardial infraction, pain, in particular neuropathic pain.

The compounds of the present invention may also be used to inhibit the entry of Human Immunodeficiency Virus (HIV) into monocytes and lymphocytes, thereby having a therapeutic role in the treatment of AIDS (Acquired Immunodeficiency Syndrome).

The CCR2 receptor exists in two isoforms, namely the CCR2A and the CCR2B receptor.

Due to their CCR2 receptor antagonistic activity, in particular their CCR2B receptor antagonistic activity, the compounds of formula (I), their N-oxides, pharmaceutically acceptable addition salts, quaternary amines, polymorphic forms or stereochemically isomeric forms are useful in the treatment or prevention, in particular for the treatment, of diseases or conditions mediated through the activation of the CCR2 receptor, in particular the CCR2B receptor. Diseases or conditions related to an activation of the CCR2 receptor comprise inflammatory conditions such as arthritis, osteoarthritis, rheumatoid arthritis, glomerulonephritis, diabetic nephropathy, lung fibrosis, idiopathic pulmonary fibrosis, sarcoidosis, vasculitis, hepatitis, nonalcoholic steatohepatitis, inflammatory conditions of the brain such as Alzheimer's disease, restenosis, alveolitis, asthma, allergic rhinitis, allergic conjunctivitis, atherosclerosis, psoriasis, delayed-type hypersensitivity reactions of the skin, inflammatory bowel disease, acute or chronic brain inflammation, e.g. multiple sclerosis, autoimmune encephalomyelitis, chronic obstructive pulmonary disease (COPD), uveitis, dermatitis, atopic dermatitis, autoimmune diseases such as diabetes or transplant rejection, stroke, reperfusion injury, ischemia, cancer, myocardial infraction, pain (neuropathic pain). In particular, the compounds of formula (I) are useful in the treatment or prevention of inflammatory diseases and autoimmune diseases, especially rheumatoid arthritis, atherosclerosis, multiple sclerosis, inflammatory bowel disease and chronic obstructive pulmonary disease (COPD). The compounds of formula (I) are also of particular interest in the treatment or prevention of psoriasis, asthma, rheumatoid arthritis or pain (neuropathic pain), more in particular psoriasis, asthma or rheumatoid arthritis.

In view of the above-described pharmacological properties, the compounds of formula (I), their N-oxides, pharmaceutically acceptable addition salts, quaternary amines and stereochemically isomeric forms, may be used as a medicine. In particular, the present compounds can be used for the manufacture of a medicament for treating or preventing diseases mediated through activation of the CCR2 receptor, in particular the CCR2B receptor. More in particular, the compounds of the invention can be used for the manufacture of a medicament for treating or preventing inflammatory diseases, especially rheumatoid arthritis, atherosclerosis, multiple sclerosis, inflammatory bowel disease and chronic obstructive pulmonary disease (COPD). The compounds of the invention can also in particular be used for the manufacture of a medicament for treating or preventing psoriasis, asthma, rheumatoid arthritis or pain (neuropathic pain), more in particular psoriasis, asthma or rheumatoid arthritis.

In view of the utility of the compounds of formula (I), there is provided a method of treating warm-blooded animals, including humans, suffering from or a method of preventing warm-blooded animals, including humans, to suffer from diseases mediated through activation of the CCR2 receptor, in particular mediated through the CCR2B receptor. Said methods comprise the administration of an effective amount of a compound of formula (I), a N-oxide form, a pharmaceutically acceptable addition salt, a quaternary amine, a polymorphic form or a possible stereoisomeric form thereof, to warm-blooded animals, including humans.

The blockade of the CCR2 receptor by the present compounds of formula (I) inhibits the normal function of MCP-1. Therefore, the present compounds can also be described as MCP-1 inhibitors and hence can be used to prevent or treat diseases mediated through MCP-1.

The present invention also provides compositions for preventing or treating diseases mediated through activation of the CCR2 receptor, in particular the CCR2B receptor. Said compositions comprise a therapeutically effective amount of a compound of formula (I) and a pharmaceutically acceptable carrier or diluent.

The compounds of the present invention may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, particularly, for administration orally, rectally, percutaneously, or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules, and tablets. Because of their ease in administration, tablets and capsules represent the most advantageous oral dosage unit forms, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations, which are intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

The compounds of the present invention may also be administered via inhalation or insufflation by means of methods and formulations employed in the art for administration via this way. Thus, in general the compounds of the present invention may be administered to the lungs in the form of a solution, a suspension or a dry powder. Any system developed for the delivery of solutions, suspensions or dry powders via oral or nasal inhalation or insufflation are suitable for the administration of the present compounds.

The compounds of the present invention may also be topically administered in the form of drops, in particular eye drops. Said eye drops may be in the form of a solution or a suspension. Any system developed for the delivery of solutions or suspensions as eye drops are suitable for the administration of the present compounds.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof.

The exact dosage and frequency of administration depends on the particular compound of formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

The compounds of formula (I) may also be used in combination with other conventional anti-inflammatory or immunosuppressive agents, such as steroids, cyclooxygenase-2 inhibitors, non-steroidal-anti-inflammatory drugs, TNF-α antibodies, such as for example acetyl salicylic acid, bufexamac, diclofenac potassium, sulindac, diclofenac sodium, ketorolac trometamol, tolmetine, ibuprofen, naproxen, naproxen sodium, tiaprofen acid, flurbiprofen, mefenamic acid, nifluminic acid, meclofenamate, indomethacin, proglumetacine, ketoprofen, nabumetone, paracetamol, piroxicam, tenoxicam, nimesulide, fenylbutazon, tramadol, beclomethasone dipropionate, betamethasone, beclamethasone, budesonide, fluticasone, mometasone, dexamethasone, hydrocortisone, methylprednisolone, prednisolone, prednisone, triamcinolone, celecoxib, rofecoxib, valdecoxib, infliximab, leflunomide, etanercept, CPH 82, methotrexate, sulfasalazine, antilymphocytory immunoglobulins, antithymocytory immunoglobulins, azathioprine, cyclosporine, tacrolimus substances, ascomycin, rapamycin, muromonab-CD3.

Thus, the present invention also relates to the combination of a compound of formula (I) and another anti-inflammatory or immunosuppressive agent. Said combination may be used as a medicine. The present invention also relates to a product containing (a) a compound of formula (I), and (b) another anti-inflammatory or immunosuppressive compound, as a combined preparation for simultaneous, separate or sequential use in the treatment of diseases mediated through activation of the CCR2 receptor, in particular mediated through the CCR2B receptor. The different drugs in such products may be combined in a single preparation together with pharmaceutically acceptable carriers. Alternatively, such products may comprise, for example, a kit comprising a container with a suitable composition containing a compound of formula (I) and another container with a composition containing another anti-inflammatory or immunosuppressive compound. Such a product may have the advantage that a physician can select on the basis of the diagnosis of the patient to be treated the appropriate amounts of each component and the sequence and timing of the administration thereof.

The following examples are intended to illustrate the present invention.

### EXPERIMENTAL PART

Hereinafter, “THF” means tetrahydrofuran, “DIPE” means diisopropylether, “DMF” means N,N-dimethylformamide, “CDI” means 1,1′-carbonyldiimidazole.

### A. Preparation of the Intermediate Compounds

### Example A1

a. Preparation of Intermediate 1

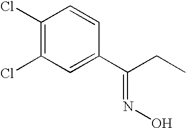

A solution of Na₂CO₃ (part of 0.52 mol) in H₂O (150 ml) was added to a stirring mixture of 1-(3,4-dichlorophenyl)-1-propanone (0.345 mol) in ethanol, p.a. (150 ml), then the remainder of Na₂CO₃ was added and hydroxylamine monohydrochloride (0.345 mol) was added portionwise while stirring vigorously. The reaction mixture was heated to reflux temperature and extra H₂O (75 ml) was added, then the resulting mixture was stirred and refluxed for 6 hours. Extra hydroxylamine monohydrochloride (2.4 g) was added and the mixture was refluxed further for 18 hours. Again extra hydroxylamine monohydrochloride (3 g) was added; the reaction mixture was refluxed for 24 hours and stirred for 2 days at room temperature. The solids were filtered off, washed with EtOH/H₂O (1/1) and dried (vacuum, stream of air) at 56° C. Yield: 71.8 g of intermediate 1 (95.4%).
b. Preparation of Intermediate 2 and 2a

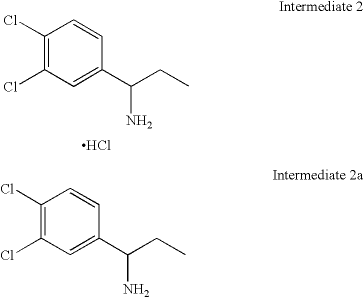

A mixture of intermediate 1 (0.3 mol) in CH₃OH/NH₃ (7 N) (500 ml) was hydrogenated at 14° C. with Raney Nickel (cat. quant.) as a catalyst in the presence of thiophene solution (6 ml). After uptake of H₂ (2 equiv.), the catalyst was filtered off and the filtrate was evaporated, then co-evaporated 2 times with toluene. The residue was stirred in boiling 2-propanol (250 ml) and the mixture was filtered off hot. The filtrate was allowed to reach room temperature and HCl/2-propanol (6N, 150 ml) was added slowly while stirring vigorously. The solvent was evaporated and the residue was stirred in DIPE, then filtered off, washed and dried (vacuum) at 60° C. Yield: 53 g intermediate 2 (73.4%). A part of this fraction was converted into its free base: Intermediate 2 (18.0 g) was stirred in CH₂Cl₂ (200 ml) and a 15% aqueous K₂CO₃ solution was added, then the resulting mixture was stirred for 1 hour and a 50% NaOH solution was added to upper the pH. The organic layer was separated, washed with H₂O, dried (MgSO₄), filtered off and the solvent was evaporated. Yield: 12.4 g of intermediate 2a.

### Example A2

a. Preparation of Intermediate 3

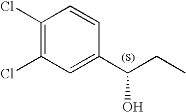

A mixture of N,N′-(1R,2R)-1,2-cyclohexanediylbis[1,1,1-trifluoromethanesulfonamide] (0.005 mol) and Ti(i-PrO)₄ (0.030 mol) in toluene (q.s.) was degassed and placed under Ar-flow, then the reaction mixture was stirred for 20 minutes at 40° C. and cooled to −78° C. Et₂Zn (0.030 mol) was added dropwise and after 20 minutes, a mixture of 3,4-dichlorobenzaldehyde (0.0250 mol) in toluene (q.s.) was added dropwise. The reaction mixture was allowed to reach 0° C. The mixture was stirred overnight at room temperature, then quenched with HCl (2N). This mixture was extracted with CH₂Cl₂. The separated organic layer was washed, dried, filtered and the solvent evaporated. The residue was purified by column chromatography over silica gel (eluent CH₂Cl₂/CH₃OH 98/2). The product fractions were collected and the solvent was evaporated. Yield: 5.1 g of intermediate 3.

The R isomer can be prepared by the above reaction by using N,N′-(1S,2S)-1,2-cyclohexanediylbis[1,1,1-trifluoromethanesulfonamide] as catalyst (see Example A3).
b. Preparation of Intermediate 4

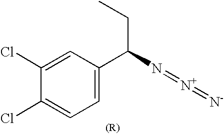

A mixture of intermediate 3 (prepared according to A2.a) (0.025 mol) and phosphorazidic acid, diphenyl ester (0.030 mol) in toluene (50 ml) was stirred at 0° C. and 2,3,4,6,7,8,9,10-octahydropyrimido[1,2-a]azepine (0.030 mol) was added. The reaction mixture was stirred for 2 hours at 0° C., then stirred overnight at room temperature. The mixture was diluted with water and toluene. The organic layer was separated, washed once with water, once with 5% HCl, and the solvent was evaporated, yielding intermediate 4, used in next reaction step.
c. Preparation of Intermediate 5

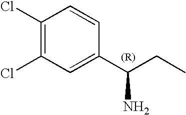

A mixture of intermediate 4 (prepared according to A2.b) (0.025 mol), triphenylphosphine (0.027 mol) in THF (70 ml) and H₂O (20 ml) was stirred overnight at room temperature. The solvent was evaporated. The residue was treated with 10% HCl. The acidic layer was washed with DIPE, then alkalized, followed by an extraction with CH₂Cl₂. The separated organic layer was dried, filtered and the solvent evaporated. The residue was purified by column chromatography over silica gel. The product fractions were collected and the solvent was evaporated. Yield: 1.1 g of intermediate 5.

### Example A3

a. Preparation of Intermediate 6

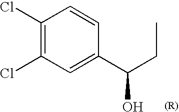

A mixture of N,N′-(1S,2S)-1,2-cyclohexanediylbis[1,1,1-trifluoro-methanesulfonamide] (0.060 g) and Ti(iPrO)₄ (8.5 g) in toluene was degassed, placed under Ar flow, then stirred for 20 minutes at 40° C. The mixture was cooled to −78° C. and diethylzinc (q.s.) was added dropwise. After 20 minutes, 3,4-dichloro-benzaldehyde (0.025 mol) in toluene (q.s.) was added dropwise and the reaction mixture was allowed to warm up to 0° C., then was stirred overnight at room temperature, quenched with 2 N HCl and extracted with CH₂Cl₂. The separated organic layer was washed, dried, filtered and the solvent evaporated. The residue was purified by column chromatography over silica gel (eluent: CH₂Cl₂/CH₃OH 98/2). The product fractions were collected and the solvent was evaporated. Yield: 5 g of intermediate 6 (R).
b. Preparation of Intermediate 7

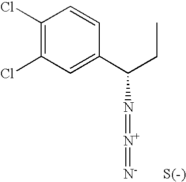

A mixture of intermediate 6 (0.127 mol) and phosphorazidic acid, diphenyl ester (0.153 mol) in toluene (q.s.) was stirred at 0° C. 2,3,4,6,7,8,9,10-octahydro-pyrimido[1,2-a]azepine (0.153 mol) was added dropwise and the reaction mixture was stirred for 1 hour at 0° C., then for 2 hours at room temperature, then for 3 hours at 50° C. The reaction mixture was cooled, washed with water, with 0.5 M HCl, with water, dried, filtered and the solvent evaporated. The residue was purified by flash column chromatography over silica gel (eluent: CH₂Cl₂/CH₃OH 99.5/0.5). The product fractions were collected and the solvent was evaporated. Yield: 23.5 g of intermediate 7.
c. Preparation of Intermediate 8

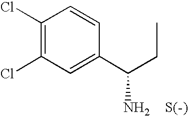

A mixture of intermediate 7 (0.122 mol) in methanol (q.s.) was hydrogenated at 50° C. with Pt/C 5% (5 g) as a catalyst. After uptake of H₂, the catalyst was filtered off and the filtrate was evaporated. Yield: intermediate 8.

### Example A4

a. Preparation of Intermediate 9

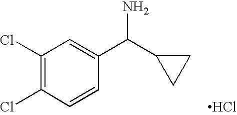

A mixture of cyclopropyl(3,4-dichlorophenyl)methanone oxime (0.16 mol) and Zn (75 g) in acetic acid (750 ml) was stirred at room temperature for 18 hours, then the reaction mixture was filtered over celite and the filtrate was evaporated. The residue was stirred in H₂O and dissolved, then the solution was treated with Na₂CO₃ and extracted with CH₂Cl₂. The organic layer was separated, dried, filtered and the solvent was evaporated. The residue was dissolved in 2-propanol and converted into the hydrochloric acid salt (1:1) with HCl/2-propanol. The precipitate was filtered off, washed with DIPE, then dried. Yield: 26.8 g of intermediate 9.
b. Preparation of Intermediate 10 and 11

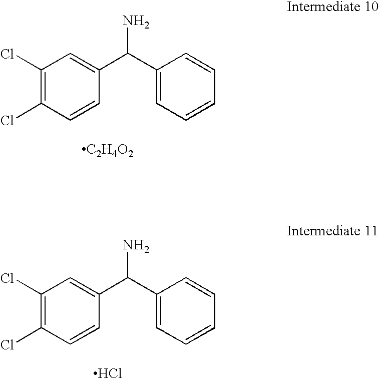

A mixture of (3,4-dichlorophenyl)phenylmethanone oxime (0.132 mol) and Zn (70 g) in acetic acid (700 ml) was stirred at room temperature for 18 hours, then the reaction mixture was filtered over decalite (to remove Zn) and the filtrate was evaporated. The residue was dissolved in H₂O (±150 ml) and converted into the acetic acid salt (1:1). The precipitate was filtered off and dried. Yield: 31 g of intermediate 10. The filtrate was treated with Na₂CO₃ and extracted with CH₂Cl₂. The organic layer was separated, dried, filtered and the solvent was evaporated. The residue was dissolved in 2-propanol and converted into the hydrochloric acid salt (1:1) with HCl/2-propanol. The precipitate was filtered off and dried. Yield: 5 g of intermediate 11.

### Example A5

a. Preparation of Intermediate 12

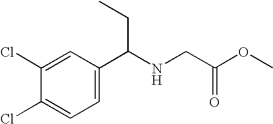

A solution of intermediate 2 (prepared according to A1.b) (0.0748 mol) and chloro acetic acid methyl ester (0.08 mol) in DMF, p.a., dried on molecular sieves, (150 ml) was stirred at room temperature under N₂ and Et₃N (0.224 mol) was slowly added, then the reaction mixture was stirred for 20 hours at room temperature and extra chloro acetic acid methyl ester (3.3 ml) was added. The mixture was stirred for another 20 hours at room temperature and again extra chloro acetic acid methyl ester (2 ml) was added. The resulting mixture was stirred for 24 hours and then the solids were filtered off and washed with DMF. Et₂O (800 ml) was added and the mixture was washed 3 times with H₂O (500 ml). The organic layer was separated, dried (MgSO₄), filtered off and the solvent was evaporated, then co-evaporated with toluene. The residual oil (23.4 g) was filtered over silica gel (eluent: CH₂Cl₂/CH₃OH 99/1). The product fractions were collected and the solvent was evaporated, finally co-evaporated with toluene. Yield: 20.6 g of intermediate 12 (99.7%).
b. Preparation of Intermediate 13

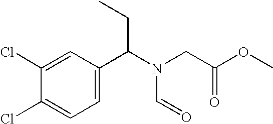

A solution of formic acid (7.5 ml) and intermediate 12 (0.0746 mol) in xylene, p.a. (225 ml) was stirred and refluxed for 4 hours and then the reaction mixture was allowed to reach room temperature. The mixture was washed 2 times with H₂O (2×200 ml), with a saturated aqueous NaHCO₃ solution (200 ml) and with brine (200 ml), then the separated organic layer was dried (MgSO₄) and filtered off. Finally, the solvent was evaporated. Yield: 21.3 g of intermediate 13 (93.9%)

### Example A6

a. Preparation of Intermediate 14

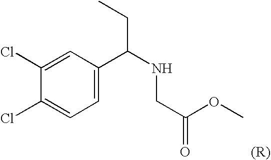

A mixture of intermediate 5 (prepared according to A2.c) (0.0054 mol), bromo-acetic acid methyl ester (0.0055 mol) and Et₃N (0.006 mol) in DMF (q.s.) was stirred overnight at room temperature, then poured out into water. This mixture was extracted with CH₂Cl₂. The separated organic layer was dried, filtered and the solvent evaporated. Yield: 1.3 g of intermediate 14 (R-isomer).
b. Preparation of Intermediate 15

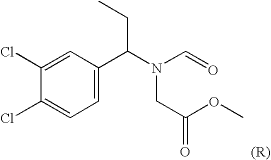

A mixture of intermediate 14 (0.0054 mol) in formic acid (3 ml) and xylene (50 ml) was stirred and refluxed for 20 hours. The reaction mixture was cooled, washed with water, dried, filtered and the solvent evaporated. Yield: 1.3 g of intermediate 15 (R-isomer).

### Example A7

a. Preparation of Intermediate 16

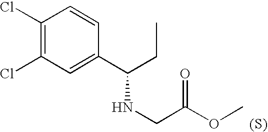

A mixture of intermediate 8 (0.050 mol), methyl bromoacetate (0.060 mol) and Et₃N (15 ml) in DMF (100 ml) was stirred overnight at room temperature. More methyl bromoacetate was added, and the reaction mixture was stirred overnight at room temperature, then poured out into water. This mixture was extracted with CH₂Cl₂. The separated organic layer was dried, filtered and the solvent evaporated. Yield: 12 g of intermediate 16.
c. Preparation of Intermediate 17

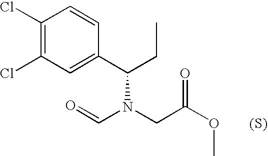

A mixture of intermediate 16 (0.05 mol) in formic acid (100 ml) and xylene (150 ml) was stirred and refluxed for 48 hours. The reaction mixture was cooled, poured out into water, then extracted with toluene. The separated organic layer was washed with water, treated with NaHCO₃, dried, filtered and the solvent evaporated. Yield: 13.2 g of intermediate 17.

### Example A8

Preparation of Intermediate 18

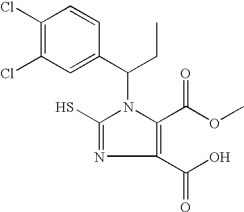

A solution of intermediate 13 (0.0618 mol) and ethanedioic acid dimethyl ester (0.11 mol) in THF (p.a., dried on molecular sieves) (100 ml) was stirred under N₂-atm., then 2-methyl-2-propanol sodium salt (0.066 mol) was added and the reaction mixture was stirred at room temperature for 18 hours and another for 24 hours. Finally the mixture was stirred at 60° C. for 4 hours. Extra 2-methyl-2-propanol sodium salt (4 g) and extra ethanedioic acid dimethyl ester (6 g) were added and the reaction mixture was stirred over the weekend at room temperature. The solvent was evaporated, the residue was dissolved in H₂O (250 ml) and washed 2 times with Et₂O. The aqueous layer was separated and CH₃OH (200 ml), KSCN (10 g) and HCl (36%, p.a.) (q.s.) were added, then the mixture was stirred for 18 hours at 60° C. The solvent was partly evaporated and the concentrate was extracted with CH₂Cl₂. The organic layer was separated, dried (MgSO₄), filtered and the solvent was evaporated. The residue (5 g) was purified by filtration over silica gel (eluent: CH₂Cl₂/CH₃OH 99/1). The desired fractions were combined and the solvent was evaporated, then co-evaporated with Hexane/DIPE. The residue was stirred in Et₂O/Hexane and the resulting precipitate was filtered off, washed with hexane, then dried (vac., 50° C.). Yield: 0.28 g of intermediate 18.

### Example A9

a. Preparation of Intermediate 19

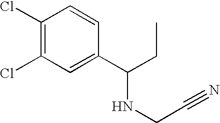

A solution of intermediate 2 (0.0116 mol) in Et₃N (0.013 mol) and DMF p.a. (20 ml) was stirred on an ice bath. A solution of chloroacetonitrile (0.0128 mol) in DMF p.a. (2.5 ml) was added dropwise. The reaction mixture was stirred at room temperature for 6 hours. Three more portions of chloroacetonitrile were added over the next 68 hours until the reaction was complete. The precipitate was filtered off. The filtrate was poured out into Et₂O (200 ml) and washed with H₂O/NaHCO₃ (10%; 100 ml) and H₂O (2×). The separated organic layer was dried (MgSO₄), filtered and the solvent was evaporated and co-evaporated with toluene. The residue was purified over silica gel (eluent: CH₂Cl₂/MeOH 99:1). The desired fractions were collected and the solvent was evaporated and co-evaporated with toluene. Yield: 2.3 g of intermediate 19 (81.6%).
b. Preparation of Intermediate 20

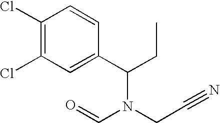

A mixture of intermediate 19 (0.00946 mol) and n-butyl-formate (15 ml) was stirred and refluxed for 4 days. The solvent was evaporated, then co-evaporated with toluene. Yield: 2.68 g of intermediate 20.
c. Preparation of Intermediate 21

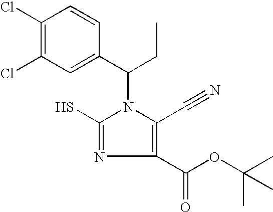

A solution of intermediate 20 (0.0158 mol) in THF p.a. dried on molecular sieves (60 ml) was stirred under N₂ and then ethanedioic acid, bis(1,1-dimethylethyl) ester (0.0238 mol) was added followed by 2-methyl-2-propanol sodium salt (0.019 mol). The reaction mixture was stirred for 4 hours at room temperature and extra 2-methyl-2-propanol sodium salt (0.4 g) was added. The mixture was stirred for 2 hours at room temperature and the solvent was evaporated. The residue was dissolved in CH₃OH (40 ml) and a solution of thiocyanic acid, potassium salt (0.0474 mol) in H₂O (20 ml) was added, then HCl 36% (2 ml) was added and the reaction mixture was stirred for 18 hours at room temperature. The mixture was further stirred at 50° C. for 24 hours, poured out into ice-water (150 ml) and extracted with CH₂Cl₂. The organic layer was separated, dried (MgSO₄), filtered off and the solvent was evaporated. The residue was purified by column chromatography over silica gel (eluent: CH₂Cl₂/CH₃OH 99.5/0.5). The product fractions were collected and the solvent was evaporated. The residue was purified by reversed phase high-performance liquid chromatography. The product fractions were collected and the organic solvent was evaporated. Precipitation occurred, so the aqueous concentrate was filtered. The filtrate was extracted with CH₂Cl₂, dried (MgSO₄), filtered off and the solvent was evaporated. The residue was stirred in DIPE, then the resulting solids were filtered off, washed and dried (vac.) at 50° C. Yield: 0.28 g of intermediate 21, melting point 172.7-175.2° C.
d. Preparation of Intermediate 22

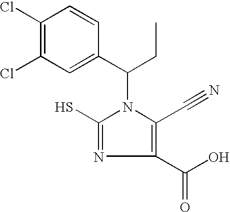

Trifluoroacetic acid (2 ml) was added to a stirring solution of intermediate 21 (0.0015 mol) in CH₂Cl₂ p.a. (25 ml), then the reaction mixture was stirred for 18 hours at room temperature (precipitation) and left to stand for 24 hours. The resulting precipitate was filtered off, washed with a small amount of CH₂Cl₂ and a lot of DIPE and finally dried (vacuum) at 50° C. Yield: 0.45 g of intermediate 22.

### Example A10

a. Preparation of Intermediate 23

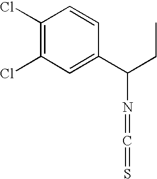

N-ethyl-N-(1-methylethyl)-2-propanamine (0.1 mol) was added to a stirring mixture of intermediate 2a (0.0415 mol) in CH₂Cl₂, p.a. (100 ml) under N₂. After 15 minutes of stirring, the reaction mixture was put on an ice bath and a solution of carbonothioic dichloride (0.0457 mol) in CH₂Cl₂, p.a. (15 ml) was added dropwise at 0° C. The mixture was stirred at 0° C. for 30 minutes and at room temperature for 18 hours, then extra N-ethyl-N-(1-methylethyl)-2-propanamine (9 ml) was added and the resulting mixture was stirred for 2 hours. The mixture was washed 2 times with H₂O, once with HCl (1N) and again with H₂O. The organic layer was separated, dried (MgSO₄), filtered off and the solvent was evaporated, then co-evaporated with toluene. The residue was purified by column chromatography over silica gel (eluent: CH₂Cl₂/Hexane 15/85). The product fractions were collected and the solvent was evaporated. Yield: 7.4 g of intermediate 23 (72.4%).
b. Preparation of Intermediate 24

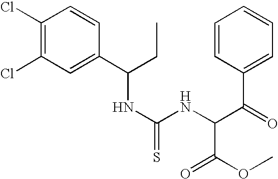

Beta-oxo-phenylalanine methyl ester monohydrochloride (0.00175 mol), followed by K₂CO₃ (0.00175 mol) and then H₂O (5 ml) were added to a solution of intermediate 23 (0.00175 mol) in THF (20 ml) and the reaction mixture was stirred at room temperature for 18 hours. The mixture was poured out into H₂O (50 ml) and extracted with CH₂Cl₂. The organic layer was separated, dried (MgSO₄), filtered off and the solvent was evaporated. The residue was purified by flash column chromatography (eluent: CH₂Cl₂/CH₃OH). The product fractions were collected and the solvent was evaporated. Yield: 0.095 g of intermediate 24 (12.4%).
c. Preparation of Intermediate 25

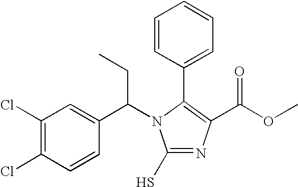

A solution of intermediate 24 (0.0002 mol) in acetic acid (6 ml) was stirred for 18 hours in a sealed tube at 100° C., then the reaction mixture was allowed to reach room temperature and was poured out into H₂O. CH₂Cl₂ was added, then a saturated K₂CO₃ solution was added until a clear biphasic solution was formed. The organic layer was separated, dried (MgSO₄), filtered off and the solvent was evaporated, then co-evaporated with toluene. The residue was purified by high-performance liquid chromatography over RP-18 (eluent: (10% NH₄OAc in H₂O)/CH₃OH/CH₃CN). The product fractions were collected and the solvent was evaporated for 50%. The concentrate was extracted with CH₂Cl₂ and the separated organic layer was evaporated. Yield: 0.011 g of intermediate 25.

### B. Preparation of the Final Compounds

### Example B1

Preparation of Compound 1

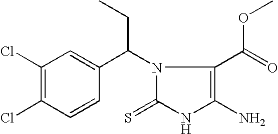

A mixture of intermediate 18 (0.0025 mol) in t-BuOH (10 ml) was stirred at room temperature and then Et₃N (0.00375 mol) and diphenyl phosphorazidic acid (0.00325 mol) were added. The reaction mixture was stirred for 20 hours at 85° C. and the solvent was evaporated under a stream of N₂ at 50° C. The obtained residue was stirred in a mixture of 2-propanol (10 ml) and HCl/2-propanol (2 ml) for 90 minutes at 80-90° C. and then the solvent was evaporated. The residue was dissolved in H₂O, treated with NaHCO₃ and extracted with CH₂Cl₂/CH₃OH (90/10). The organic layer was separated, dried, filtered off and the solvent was evaporated. The residue was filtered over silica gel (eluent: CH₂Cl₂/CH₃OH 98/2). The product fractions were collected and the solvent was evaporated. The residue was stirred in DIPE, then the resulting precipitate was filtered off and dried. Yield: 0.168 g of compound 1 (m.p.: 183.9-184° C.).

### Example B2

Preparation of Compound 2

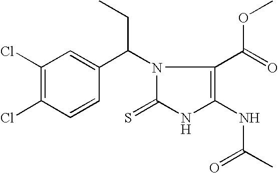

A mixture of compound 1 (prepared according to B1) (0.00028 mol) in acetyl acetate (2 ml) was stirred for 2 hours at 90° C. and then the solvent was evaporated. The residue was stirred in H₂O, extracted with CH₂Cl₂ and treated with NaHCO₃. The organic layer was separated, dried, filtered off and the solvent was evaporated. The obtained residue was filtered over silica gel (eluent: CH₂Cl₂/CH₃OH 99/1). The product fractions were collected and the solvent was evaporated. The residue was filtered again over silica gel (eluent: CH₂Cl₂/CH₃OH 99/1). The product fractions were collected and then the solvent was evaporated and the residue was dried. Yield: 0.018 g of compound 2.

### Example B3

Preparation of Compound 3

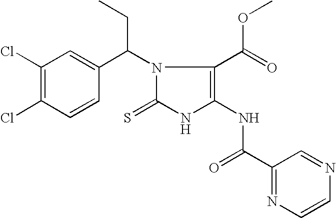

A mixture of pyrazinecarboxylic acid (0.0005 mol), N′-(ethylcarbonimidoyl)-N,N-dimethyl-1,3-propanediamine (0.0005 mol) and 1-hydroxy-1H-benzotriazole (0.0005 mol) in DMF (5 ml) was stirred for 15 minutes at room temperature and compound 1 (prepared according to B1) (0.0005 mol) was added, then the reaction mixture was stirred for 3 hours at room temperature and stirred for 18 hours at 60° C. Extra pyrazinecarboxylic acid (0.0005 mol), N′-(ethylcarbonimidoyl)-N,N-dimethyl-1,3-propanediamine (0.0005 mol) and 1-hydroxy-1H-benzotriazole (0.0005 mol) were added, followed by N,N-dimethyl-4-pyridinamine (0.001 mol) and the resulting mixture was stirred for 5 hours at room temperature. After stirring the mixture for 38 hours at 60° C., it was poured out into H₂O. The formed precipitate was filtered off and washed with H₂O, then stirred in H₂O and extracted with CH₂Cl₂. The organic layer was separated, dried, filtered off and the solvent was evaporated. The residue was filtered over silica gel (eluent: CH₂Cl₂/CH₃OH 99/1). The product fractions were collected and the solvent was evaporated. The residue was stirred in DIPE, then the resulting precipitate was filtered off and dried. Yield: 55 mg of compound 3 (m.p.: 196.9-198.6° C.).

### Example B4

Preparation of Compound 4

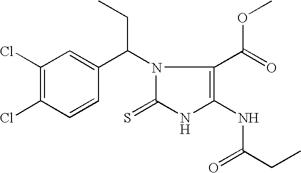

A mixture of compound 1 (5 mM), propionylchloride (15 mM) and triethylamine (15 mM) in 20 ml CH₂Cl₂ were stirred for 2 hours at 0° C. The solvent was evaporated and the residue was purified by reversed phase chromatography. The pure fractions were collected and the solvent was evaporated. The residue was dried. Yield: 10 mg of compound 4.

### Example B5

Preparation of Compound 13

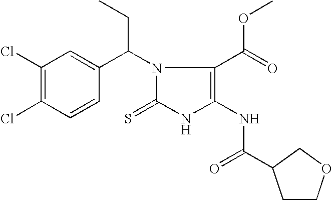

A mixture of tetrahydro-3-furancarboxylic acid (0.001 mol), N′-(ethylcarbonimidoyl)-N,N-dimethyl-1,3-propanediamine (0.001 mol), 3-oxide-1H-benztriazole (0.001 mol) and N,N-dimethylformamide (7 ml) was stirred for 15 minutes at room temperature. N,N-dimethyl-4-pyridinamine (0.001 mol) and final compound 1 (prepared according to B1) (0.0005 mol) were added and the mixture was stirred for 20 hours at 60° C. The mixture was poured out into water. The mixture was extracted with toluene. The organic layer was separated, dried, filtered and the solvent was evaporated. The residue was purified over silica gel on a glass filter (eluent: CH₂Cl₂/CH₃OH 99/1). The product fractions were collected and the solvent was evaporated. The residue was stirred in DIPE. The precipitate was filtered off and dried. Yield: 0.046 g of final compound 13.

### Example B6

Preparation of Compound 14

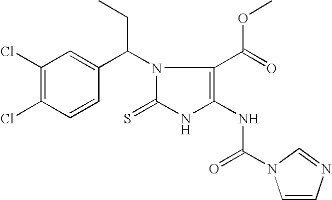

A mixture of final compound 1 (prepared according to B1) (0.0027 mol), CDI (0.01 mol) and CH₂Cl₂ (25 ml) was stirred at room temperature for 20 hours under N₂. The solvent was evaporated. The crude residue was used. Yield: 1.22 g of final compound 14.

### Example B7

a) Preparation of Compound 15

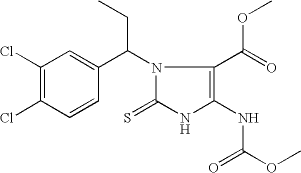

Reaction under N₂ flow. A mixture of final compound 14 (prepared according to B6) (0.0005 mol) and CH₃OH (3 ml) was stirred for 3 hours at room temperature. The solvent was evaporated. The residue was purified over silica gel on a glass filter (eluent: CH₂Cl₂/CH₃OH 99/1). The product fractions were collected and the solvent was evaporated. Yield: 0.070 g of final compound 15.
b) Preparation of Compound 16

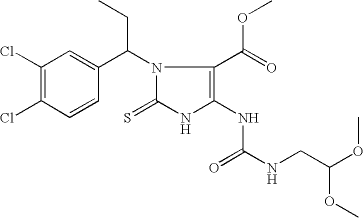

A mixture of final compound 14 (prepared according to B6) (0.00025 mol), 2,2-dimethoxyethanamine (0.003 mol) and THE (5 ml) was stirred for 20 hours at room temperature. The solvent was evaporated. The residue was purified by revered-phase high performance liquid chromatography. The product fractions were collected and the solvent was evaporated. The residue was dried. Yield: 0.013 g of final compound 16.
c) Preparation of Compound 17

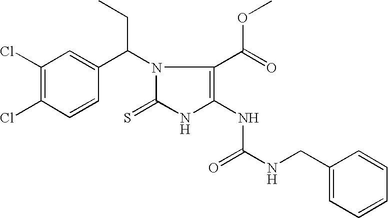

Reaction under N₂ flow. A mixture of final compound 14 (prepared according to B6) (0.0005 mol), benzenemethanamine (0.005 mol) and THF (3 ml) was stirred for 3 hours at room temperature. The solvent was evaporated. The residue was purified two times over silica gel on a glass filter (eluent gradient: CH₂Cl₂/CH₃OH 98/2 and 99/1). The product fractions were collected and the solvent was evaporated. The residue was purified by reversed-phase high performance liquid chromatography. The product fractions were collected and the solvent was evaporated. The residue was dried. Yield: 0.005 g of final compound 17.
d) Preparation of Compound 18

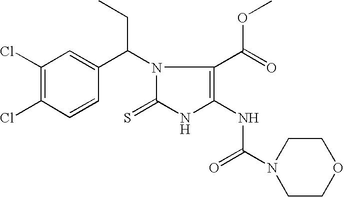

A mixture of final compound 14 (prepared according to B6) (0.00025 mol), morpholine (0.003 mol) and THF (3 ml) was stirred for 20 hours at room temperature. The solvent was evaporated. The residue was purified by reversed-phase high performance liquid chromatography. The product fractions were collected and the solvent was evaporated. The residue was dried. Yield: 0.040 g of final compound 18.

Table 1 list the compounds of formula (I) which were prepared according to one of the above examples (Ex. No.)

TABLE 1

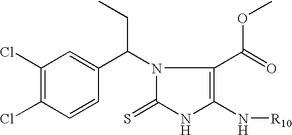

| Comp. | Exp. | | Physical data (m.p. = |
|---|---|---|---|
| No. | No. | R₁₀ | melting point) |
| 1 | B1 | H | m.p. 183.9-184° C. |
| 2 | B2 | 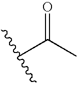 | |
| 3 | B3 | 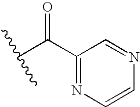 | m.p. 196.9-198.6° C. |
| 4 | B4 | 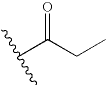 | |
| 5 | B3 | 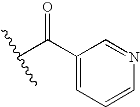 | |
| 6 | B3 | 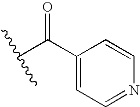 | m.p. 200-200.1° C. |
| 7 | B4 | 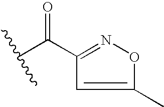 | m.p. 182.3-182.4° C. |
| 8 | B3 | 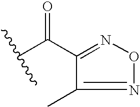 | |
| 9 | B3 | 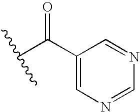 | m.p. 186.3-187.4° C. |
| 10 | B4 | 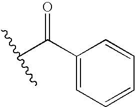 | |
| 11 | B4 | 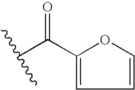 | |
| 12 | B3 | 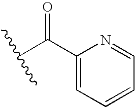 | |
| 13 | B5 | 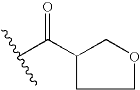 | m.p. 124.6-125.5° C. |
| 19 | B5 | 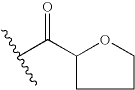 | m.p. 156.2-157.3° C. |
| 14 | B6 | 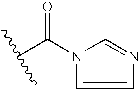 | |
| 15 | B7.a | 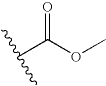 | |
| 16 | B7.b | 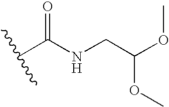 | |
| 17 | B7.c | 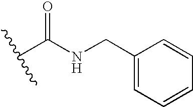 | |
| 18 | B7.d | 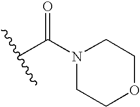 | |
| 20 | B7.d | 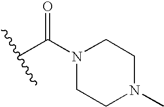 | |
| 21 | B7.d | 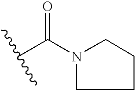 | |
| 22 | B7.d | 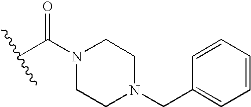 | |
| 23 | B7.d | 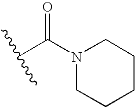 | |

### C. Analytical Part

LCMS Conditions

The HPLC gradient was supplied by a Waters Alliance HT 2790 system with a columnheater set at 40° C. Flow from the column was split to a Waters 996 photodiode array (PDA) detector and a Waters-Micromass ZQ mass spectrometer with an electrospray ionization source operated in positive and negative ionization mode. Reversed phase HPLC was carried out on a Xterra MS C18 column (3.5 μm, 4.6×100 mm) (12 minutes column) with a flow rate of 1.6 ml/minutes. Three mobile phases (mobile phase A: 95% 25 mM ammoniumacetate+5% acetonitrile; mobile phase B: acetonitrile; mobile phase C: methanol) were employed to run a gradient condition from 100% A to 50% B and 50% C in 6.5 minutes, to 100% B in 1 minute, 100% B for 1 minute and reequilibrate with 100% A for 1.5 minute. An injection volume of 10 μL was used.

Mass spectra were acquired by scanning from 100 to 1000 in is using a dwell time of 0.1 s. The capillary needle voltage was 3 kV and the source temperature was maintained at 140° C. Nitrogen was used as the nebulizer gas. Cone voltage was 10 V for positive ionization mode and 20 V for negative ionization mode. Data acquisition was performed with a Waters-Micromass MassLynx-Openlynx data system.

TABLE 2
LCMS parent peak ([M⁺] defines the exact
mass of the compound) and retention time (minutes)

| Comp. No. | [M+] | Retention time |
|---|---|---|
| 2 | 401 | 5.79 |
| 4 | 415 | 1.10 |
| 8 | 469 | 5.80 |
| 10 | 463 | 6.28 |
| 11 | 453 | 6.03 |
| 12 | 464 | 6.29 |
| 18 | 473 | 5.85 |
| 23 | 471 | 6.35 |
| 22 | 562 | 6.58 |
| 21 | 457 | 7.13 |
| 20 | 586 | 5.88 |
| 17 | 491 | 6.53 |
| 15 | 418 | 5.77 |
| 16 | 491 | 5.5 |

### D. Pharmacological Example

Inhibition of MCP-1 Induced Ca-flux in Human THP-1 Cells

MCP-1 binding to the CCR2 receptor induces a rapid and transient intracellular release of Ca²⁺ (secondary messenger) in several cell lines (Charo et al, PNAS 1994). Free Ca²⁺ levels can be measured using a Ca²⁺ sensitive dye. When the CCR2 receptor is blocked with a CCR2 receptor antagonist, the MCP-1 induced release of Ca²⁺ is inhibited.

Human THP-1 cells (monocytic cell line, ATCC TIB-202) were cultured in RPMI 1640 medium supplemented with 10% fetal calf serum (FCS), 1% L-Glutamine, penicillin (50 U/ml) and streptomycin (50 μg/ml) (all GIBCO BRL, Gent). After centrifugation, cells were loaded for 30 minutes with the Ca²⁺ sensitive fluorescent dye Fluo-3 AM (Molecular Probes, Leiden, Netherlands) (2 million cells/ml in RPMI medium containing 4 μM Fluo-3 AM, 20 mM HEPES, 0.1% Bovine Serum Albumin (BSA) and 5 mM probenecid). Excess dye was removed by 3-fold washing with buffer (5 mM HEPES, 140 mM NaCl, 1 mM MgCl₂, 5 mM KCl, 10 mM glucose, 2.5 mM probenecid, 1.25 mM CaCl₂, 0.1% BSA; all further incubations were done in this buffer). Cells were plated at a density of 150 000 cells/well in dark-wall 96-well plates (Costar, Cambridge, Mass.) and sedimented by centrifugation (1 minute). The cells were pre-incubated for 20 minutes with test compound. Then, 10⁻⁷ M hMCP-1 (Bachem, Bubendorf, Switserland) was added. Changes in intracellular free Ca²⁺ concentration were measured using the Fluorescent Imaging Plate Reader (FLIPR, Molecular Devices, Munchen, Germany). Fluorescence was recorded every second from 10 seconds before the addition of the MCP-1 till 2 minutes after the addition (first minute: 60 records with 1 second intervals, second minute 20 records with 3 second intervals). The maximal fluorescence obtained during this time frame was used for further calculations.

Table 3 reports pIC₅₀ values obtained in the above-described test for compounds of formula (I). pIC₅₀ defines −log IC₅₀ wherein IC₅₀ is the molar concentration of the test compound which inhibits 50% of specific MCP-1 induced Ca²⁺ flux.

TABLE 3

| Comp. No. | pIC₅₀ |
|---|---|
| 1 | 7.2 |
| 2 | 7.3 |
| 3 | 7.1 |
| 4 | 6.9 |
| 5 | 6.7 |
| 6 | 6.9 |
| 7 | 6.8 |
| 8 | 6.3 |
| 9 | 6.7 |
| 10 | 6.7 |
| 11 | 7.6 |
| 12 | 7.4 |
| 13 | 7.1 |
| 15 | 7.8 |
| 16 | 6.6 |
| 17 | 6.2 |
| 18 | 7 |
| 19 | 7.1 |
| 20 | 6.5 |
| 21 | 6.9 |
| 22 | 6.4 |
| 23 | 6.8 |

1. A compound of formula 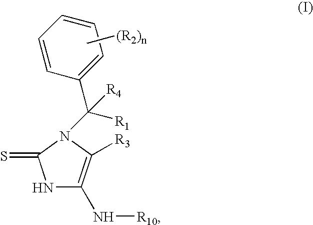
a N-oxide, a pharmaceutically acceptable addition salt, a quaternary amine or a stereochemically isomeric form thereof, wherein
R₁ represents hydrogen, C₁₋₆alkyl, C₃₋₇cycloalkyl, C₁₋₆alkyloxyC₁₋₆alkyl, di(C₁₋₆alkyl)aminoC₁₋₆alkyl, aryl or heteroaryl;
each R₂ independently represents halo, C₁₋₆alkyl, C₁₋₆alkyloxy, C₁₋₆alkylthio, polyhaloC₁₋₆alkyl, polyhaloC₁₋₆alkyloxy, cyano, aminocarbonyl, amino, mono- or di(C₁₋₄alkyl)amino, nitro, aryl or aryloxy;
R₃ represents cyano, C(═O)—O—R₅, C(═O)—NR₆ₐR_{6b} or C(═O)—R₇; or a cyclic ring system selected from 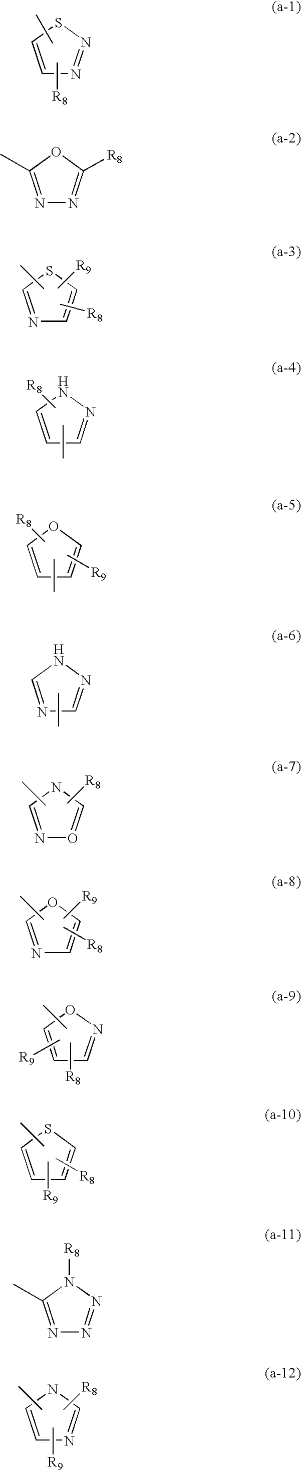 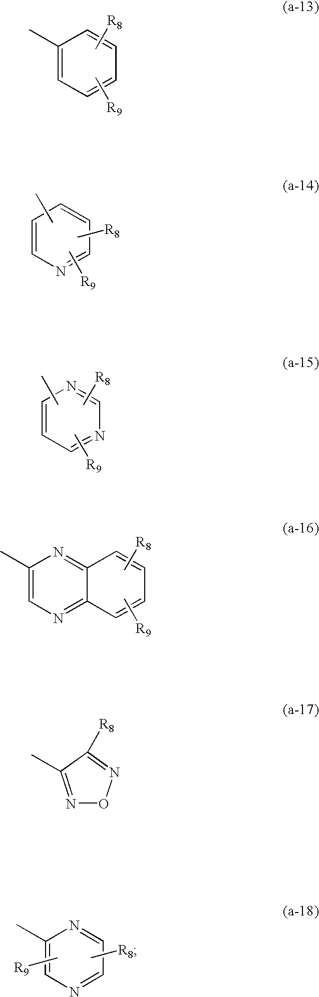
R₄ represents hydrogen or C₁₋₆alkyl;
R₅ represents hydrogen, C₁₋₆alkyl, hydroxyC₁₋₆alkyl, C₂₋₆alkenyl, C₂₋₆alkynyl, polyhaloC₁₋₆alkyl, C₁₋₆alkyloxyC₁₋₆alkyl optionally substituted with C₁₋₆alkyloxy, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl, aryl or arylC₁₋₆alkyl;
R₆ₐ and R_{6b} each independently represent hydrogen, C₁₋₆alkyl, amino, mono- or di(C₁₋₄alkyl)amino, arylNH—, aminoC₁₋₁₆alkyl, mono- or di(C₁₋₄alkyl)amino-C₁₋₆alkyl, C₁₋₆alkylcarbonylamino, aminocarbonylamino, C₁₋₆alkyloxy, carbonylamino or hydroxyC₁₋₆alkyl; or
R₆ₐ and R_{6b} taken together with the nitrogen to which they are attached form pyrrolidinyl, imidazolidinyl, pyrazolidinyl, piperidinyl, piperazinyl, morpholinyl, thiomorpholinyl or piperazinyl substituted with C₁₋₆alkyl;
R₇ represents hydrogen, C₁₋₆alkyl, hydroxyC₁₋₆alkyl, C₂₋₆alkenyl, C₂₋₆alkynyl, polyhaloC₁₋₆alkyl, C₁₋₆alkyloxyC₁₋₆alkyl, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl, aryl or heteroaryl;
each R₈ independently represents hydrogen, halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, hydroxyC₁₋₆alkylamino, aryl, aryloxy, piperidinyl, piperidinylamino, morpholinyl, piperazinyl or nitro;
each R₉ independently represents hydrogen, halo or C₁₋₆alkyl;
R₁₀ represents hydrogen, C₁₋₆alkyl, C₁₋₆alkylcarbonyl, C₁₋₆alkyloxycarbonyl, arylcarbonyl, heteroarylcarbonyl, —C(═O)—NH—R₅, —C(═S)—NH—R₅ or —S(═O)₂—R₅;
n is 1, 2, 3, 4 or 5;
aryl represents phenyl or phenyl substituted with one, two, three, four or five substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, phenyloxy or nitro;
heteroaryl represents pyrrolidinyl, tetrahydrofuranyl, imidazolidinyl, pyrazolidinyl, pyrrolinyl, imidazolinyl, pyrazolinyl, furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, piperidinyl, piperazinyl, morpholinyl, thiomorpholinyl, each of said heterocycles optionally being substituted with one or two substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, nitro or arylC₁₋₆alkyl.

2. A compound according to claim 1 wherein
R₅ represents hydrogen, C₁₋₆alkyl, hydroxyC₁₋₆alkyl, C₂₋₆alkenyl, C₂₋₆alkynyl, polyhaloC₁₋₆alkyl, C₁₋₆alkyloxyC₁₋₆alkyl, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl, aryl or arylC₁₋₆alkyl;
R₁₀ represents hydrogen, C₁₋₆alkyl, C₁₋₆alkylcarbonyl, arylcarbonyl, heteroarylcarbonyl, —C(═O)—NH—R₅, —C(═S)—NH—R₅ or —S(═O)₂—R₅;
heteroaryl represents furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, each of said heterocycles optionally being substituted with one or two substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino or nitro.

3. A compound according to claim 1 wherein the compound is a compound of formula 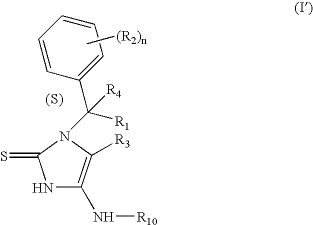
a N-oxide, a pharmaceutically acceptable addition salt, a quaternary amine or a stereochemically isomeric form thereof.

4. A compound according to claim 1, wherein R₃ represents C(═O)—O—R₅.

5. A compound according to claim 1, wherein R₁₀ represents hydrogen, C₁₋₆alkylcarbonyl, C₁₋₆alkyloxycarbonyl, arylcarbonyl, heteroarylcarbonyl, or —C(═O)—NH—R₅.

6. A compound according to claim 5 wherein R₁₀ represents C₁₋₆alkylcarbonyl, C₁₋₆alkyloxycarbonyl, arylcarbonyl, heteroarylcarbonyl, or —C(═O)—NH—R₅.

7. A compound according to claim 6 wherein R₁₀ represents C₁₋₆alkylcarbonyl, arylcarbonyl or heteroarylcarbonyl.

8. A compound according to claim 1, wherein n is 2.

9. A compound according to claim 1, wherein R² is halo.

10. A compound according to claim 1 wherein R₁ is C₁₋₆alkyl.

11. A compound according to claim 1 wherein R₄ represents hydrogen.

12. A compound according to claim 1, wherein R₅ represents C₁₋₆alkyl, arylC₁₋₆alkyl or C₁₋₆alkyloxyC₁₋₆alkyl optionally substituted with C₁₋₆alkyloxy.

13. A compound according to claim 1, wherein R₅ represents C₁₋₆alkyl, arylC₁₋₆alkyl or C₁₋₆alkyloxyC₁₋₆alkyl.

14. A compound according to claim 1 wherein
R₁ represents C₁₋₆alkyl; R₂ represents halo; R₃ represents C(═O)—O—R₅; R₁₀ represents hydrogen, C₁₋₆alkylcarbonyl, C₁₋₆alkyloxycarbonyl, —C(═O)—NH—R₅, arylcarbonyl or heteroarylcarbonyl; R₄ represents hydrogen; n is 2.

15. A compound selected from the group having the following formula: 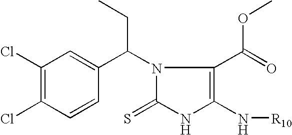
wherein R₁₀ is selected from: 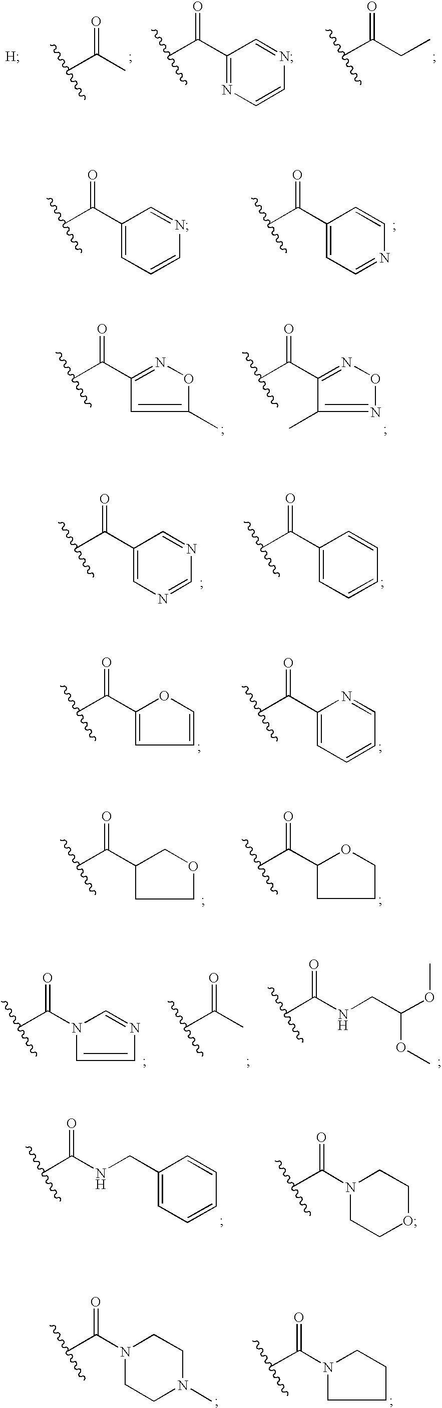 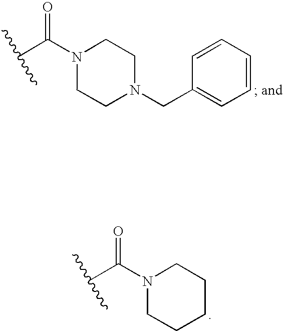
and N-oxides, pharmaceutically acceptable addition salts, quaternary amines and stereochemically isomeric forms thereof.

16. (canceled)

17. A method of preventing or treating a disease mediated through activation of the CCR2 receptor in a mammal, comprising administering an effective amount of at least one compound according to claim 1 to said mammal.

18. The method according to claim 17, wherein the disease is an inflammatory disease.

19. A pharmaceutical composition comprising a pharmaceutically acceptable carrier, and as active ingredient a therapeutically effective amount of a compound as claimed in claim 1.

20. A process of preparing a composition as claimed in claim 19, said method comprising intimately mixing a pharmaceutically acceptable carrier with a therapeutically effective amount of a compound as claimed in claim 1.

21. A process of preparing a compound as defined in claim 1, said process comprising the steps of:
a) reacting an intermediate of formula (II) with phosphorazidic acid diphenyl ester in the presence of a suitable base and a suitable solvent 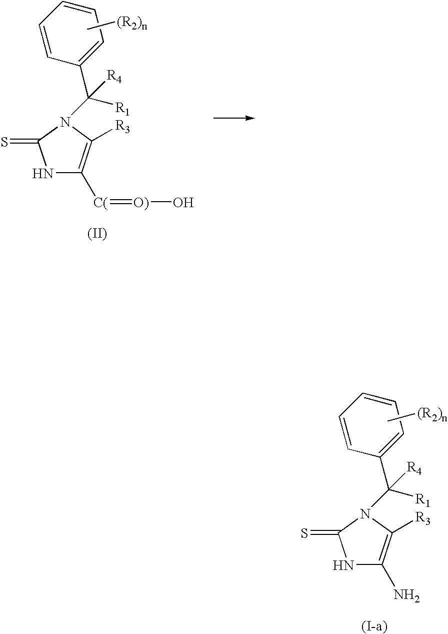
with R₁, R₂, R₃, R₄ and n as defined in claim 1;
b) reacting a compound of formula (I-a) with an intermediate of formula (III) 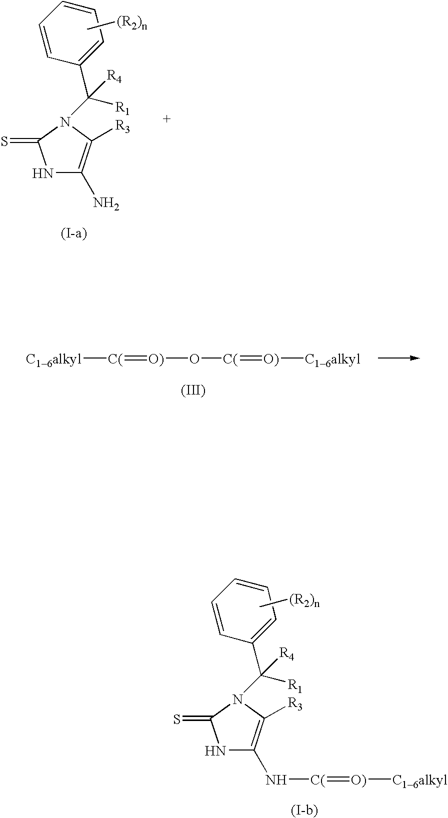
with R₁, R₂, R₃, R₄ and n as defined in claim 1;
c) reacting a compound of formula (I-a) with an intermediate of formula (IV) in the presence of suitable coupling agent, a suitable solvent, and a suitable base, 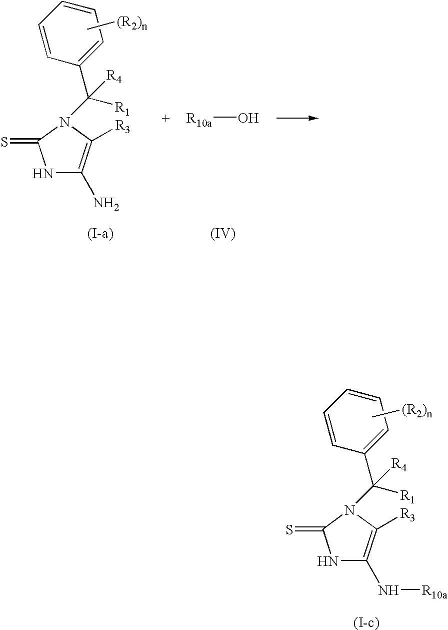
with R₁, R₂, R₃, R₄ and n as defined in claim 1 and with R₁₀ₐ representing C₁₋₆alkylcarbonyl, C₁₋₆alkyloxycarbonyl, arylcarbonyl or heterocarbonyl;
d) reacting a compound of formula (I-a) with an intermediate of formula (V) in the presence of a suitable base and a suitable solvent, 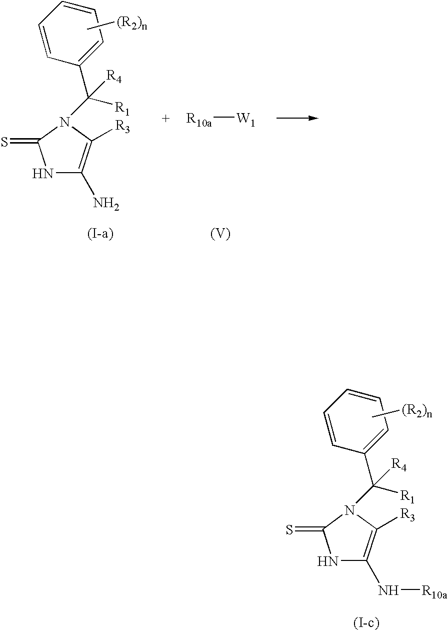
with R₁, R₂, R₃, R₄ and n as defined in claim 1, with R₁₀ₐ representing C₁₋₆alkylcarbonyl, C₁₋₆alkyloxycarbonyl, arylcarbonyl or heteroarylcarbonyl and with W₁ representing a suitable leaving group;
e) reacting a compound of formula (I-a) with 1,1′-carbonyldiimidazole in the presence of a suitable solvent, 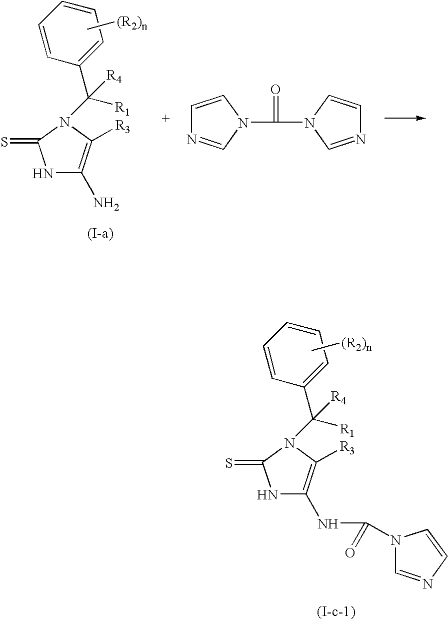
with R₁, R₂, R₃, R₄ and n as defined in claim 1;
f) reacting a compound of formula (I-c-1) with morpholine in the presence of a suitable solvent, 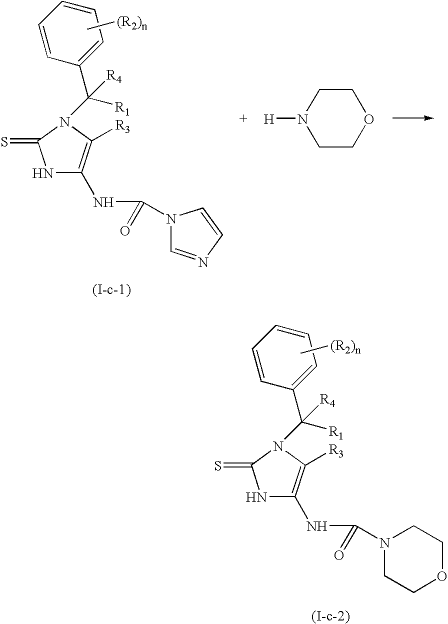
with R₁, R₂, R₃, R₄ and n as defined in claim 1;
g) reacting a compound of formula (I-c-1) with C₁₋₆alkyl-OH 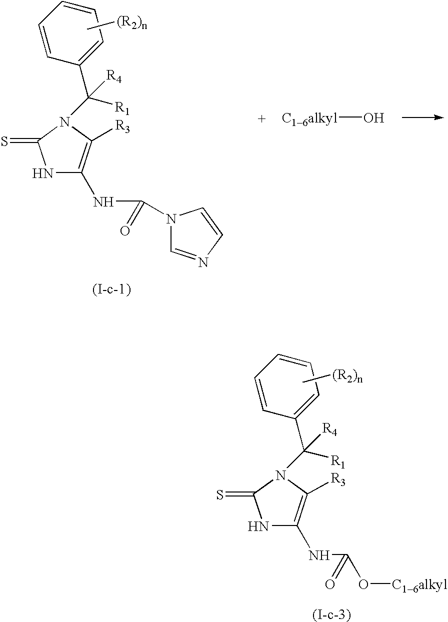
with R₁, R₂, R₃, R₄ and n as defined in claim 1;
h) reacting a compound of formula (I-a) with R₅ₐ—N═C═O respectively R₅ₐ—N═C═S in the presence of a suitable solvent and optionally in the presence of a suitable base, 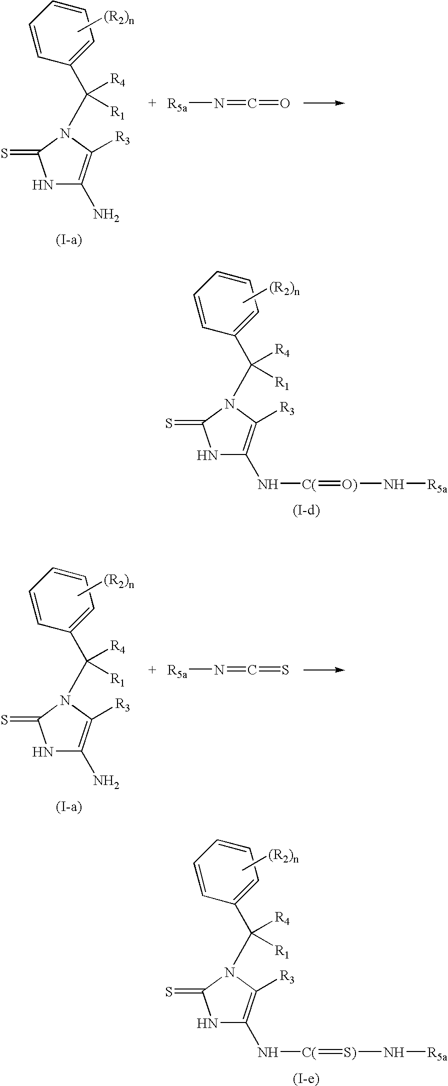
with R₁, R₂, R₃, R₄ and n as defined in claim 1 and with R₅ₐ representing R₅ as defined in claim 1 but other than hydrogen;
i) reacting a compound of formula (I-c-1) with NH₂—R_{5b} in the presence of a suitable solvent, 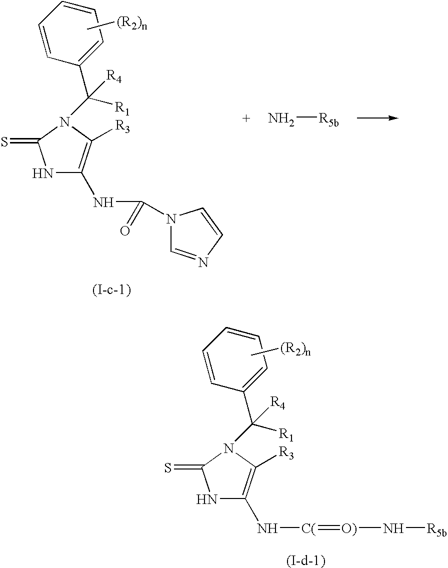
with R₁, R₂, R₃, R₄ and n as defined in claim 1 and with R_{5b} represents arylC₁₋₆alkyl or C₁₋₆alkyloxyC₁₋₆alkyl optionally substituted with C₁₋₆alkyloxy;
j) reacting a compound of formula (I-a) with W₅—N═C═O respectively W₅—N═C═S in the presence of a suitable solvent and optionally in the presence of a suitable base, followed by reaction with a suitable acid, 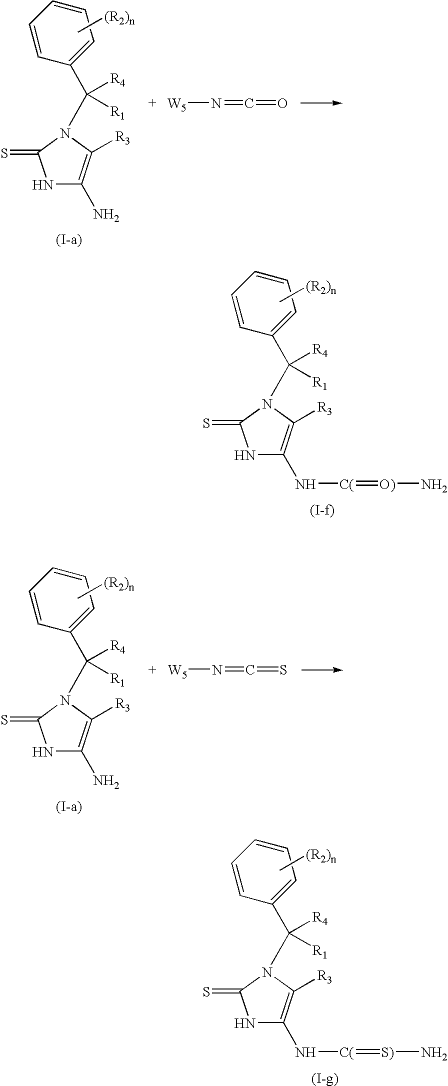
with R₁, R₂, R₃, R₄ and n as defined in claim 1 and with W₅ representing a suitable leaving group;
k) reacting a compound of formula (I-a) with an intermediate of formula W₂—S(═O)₂—R₅ in the presence of a suitable base and a suitable solvent, 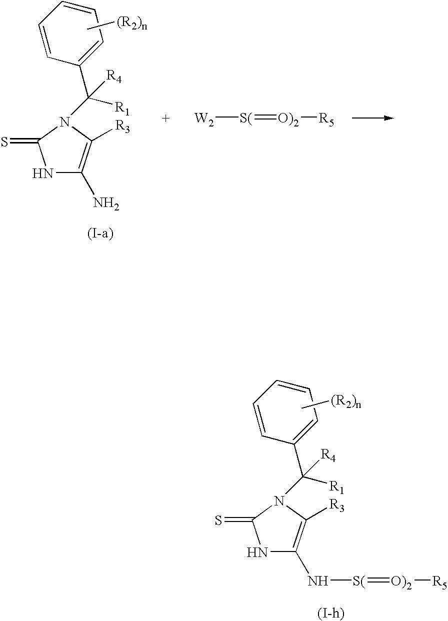
with R₁, R₂, R₃, R₄, R₅ and n as defined in claim 1 and with W₂ representing a suitable leaving group;
or, if desired, converting compounds of formula (I) into each other following art-known transformations, and further, if desired, converting the compounds of formula (I), into a therapeutically active non-toxic acid addition salt by treatment with an acid, or into a therapeutically active non-toxic base addition salt by treatment with a base, or conversely, converting the acid addition salt form into the free base by treatment with alkali, or converting the base addition salt into the free acid by treatment with acid; and, if desired, preparing stereochemically isomeric forms, quaternary amines or N-oxide forms thereof.

22. A compound according to claim 2—wherein the compound is a compound of formula 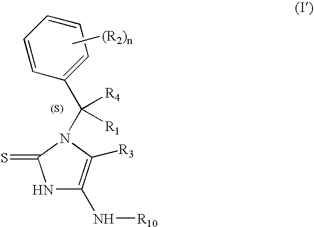
a N-oxide, a pharmaceutically acceptable addition salt, a quaternary amine or a stereochemically isomeric form thereof.

23. A compound according to claim 2, wherein R₃ represents C(═O)—O—R₅.

24. A compound according to claim 3, wherein R₃ represents C(═O)—O—R₅.

25. A compound according to claim 2,—wherein R₁₀ represents hydrogen, C₁₋₆alkylcarbonyl, C₁₋₆alkyloxycarbonyl, arylcarbonyl, heteroarylcarbonyl, or —C(═O)—NH—R₅.

26. A compound according to claim 3,—wherein R₁₀ represents hydrogen, C₁₋₆alkylcarbonyl, C₁₋₆alkyloxycarbonyl, arylcarbonyl, heteroarylcarbonyl, or —C(═O)—NH—R₅.

27. A compound according to claim 4,—wherein R₁₀ represents hydrogen, C₁₋₆alkylcarbonyl, C₁₋₆alkyloxycarbonyl, arylcarbonyl, heteroarylcarbonyl, or —C(═O)—NH—R₅.

28. A compound according to claim 2, wherein n is 2.

29. A compound according to claim 3, wherein n is 2.

30. A compound according to claim 4, wherein n is 2.

31. A compound according to claim 5, wherein n is 2.

32. A compound according to claim 6, wherein n is 2.

33. A compound according to claim 7, wherein n is 2.

34. A compound according to claim 2, wherein R² is halo.

35. A compound according to claim 3, wherein R² is halo.

36. A compound according to claim 4, wherein R² is halo.

37. A compound according to claim 5, wherein R² is halo.

38. A compound according to claim 6, wherein R² is halo.

39. A compound according to claim 7, wherein R² is halo.

40. A compound according to claim 8, wherein R² is halo.

41. A compound according to claim 9, wherein R₁ is C₁₋₆alkyl.

42. A compound according to claim 10, wherein R₄ represents hydrogen.

43. A compound according to claim 3, wherein R₅ represents C₁₋₆alkyl, arylC₁₋₆alkyl or C₁₋₆alkyloxyC₁₋₆alkyl optionally substituted with C₁₋₆alkyloxy.

44. A compound according to claim 11, wherein R₅ represents C₁₋₆alkyl, arylC₁₋₆alkyl or C₁₋₆alkyloxyC₁₋₆alkyl optionally substituted with C₁₋₆alkyloxy.

45. A compound according to claim 42, wherein R₅ represents C₁₋₆alkyl, arylC₁₋₆alkyl or C₁₋₆alkyloxyC₁₋₆alkyl optionally substituted with C₁₋₆alkyloxy.

46. A compound according to claim 12, wherein R₅ represents C₁₋₆alkyl, arylC₁₋₆alkyl or C₁₋₆alkyloxyC₁₋₆alkyl.

47. A compound according to claim 45, wherein R₅ represents C₁₋₆alkyl, arylC₁₋₆alkyl or C₁₋₆alkyloxyC₁₋₆alkyl.

48. A method of preventing or treating a disease mediated through activation of the CCR2 receptor in a mammal, comprising administering an effective amount of at least one compound according to claim 15 to said mammal.

49. The method according to claim 48, wherein the disease is an inflammatory disease.

50. A pharmaceutical composition comprising a pharmaceutically acceptable carrier, and as active ingredient a therapeutically effective amount of a compound as claimed in claim 15.
